(19) United States
(12) Patent Application Publication
(10) Pub. No.: US 2026/0034092 A1
(43) Pub. Date: Feb. 5, 2026

(54) BIOCIDAL COMPOSITION

(71) Applicant: Juan Antonio SANTISTEBAN ORTIZ, Andorra La Vella (AD)

(72) Inventor: Juan Antonio SANTISTEBAN ORTIZ, Andorra La Vella (AD)

(21) Appl. No.: 19/100,976

(22) PCT Filed: Jul. 26, 2023
(86) PCT No.: PCT/EP2023/070697

§ 371 Date: Feb. 4, 2025

(30) Foreign Application Priority Data
Aug. 4, 2022 (EP) 22382760.1

(51) Int. Cl. A61K 31/352; A61K 8/34; A61K 8/49; A61K 9/00; A61K 31/045; A61K 31/05; A61K 31/085; A61K 31/11; A61K 31/14; A61K 31/245; A61K 31/695; A61P 31/12; A61Q 15/00; A61Q 19/00; A61Q 19/08

(52) U.S. Cl. CPC A61K 31/352; A61K 8/347; A61K 8/498; A61K 9/0014; A61K 31/045; A61K 31/05; A61K 31/085; A61K 31/11; A61K 31/14; A61K 31/245; A61K 31/695; A61P 31/12; A61Q 15/00; A61Q 19/007; A61Q 19/08; A61K 2800/522; A61K 2800/524; A61K 2800/592

(57) ABSTRACT

The invention refers to compositions comprising: (a) from 25% to 43% by weight of 5-Isopropil-2-metilfenol (carvacrol); (b) from 20% to 38% by weight of 1,3,3-Trimethyl-2-oxabicyclo[2.2.2]octane (eucalyptol); and (c) from 21% to 38% by weight of 4-Methyl-1-(propan-2-yl)cyclohex-3-en-1-ol (terpinen-4-ol) being the sum of ingredients up to 100% by weight. The compositions have a strong biocidal activity. The invention also refers to products comprising the composition and also to the uses thereof.

This application claims the benefit of European Patent Application EP22382760.1 filed 4 Aug. 2022.

The present invention relates to the field of biocide compounds. Particularly, to compositions with bactericidal, virucidal, fungicidal, and yeasticidal activity; and products comprising it. It also relates to processes for their preparation and their uses in the pharmacy, veterinary, cosmetic, phytosanitary, food/feed industry, hygiene/cleaning industry and perfumery fields.

### BACKGROUND ART

Currently, bactericidal, virucidal, fungicidal, and yeasticidal agents used in the field of disinfection are of industrial chemical origin. Said agents comprise quaternary ammonium salts, ethanol and other elements that can be harmful to the environment, since in high proportions they are hardly biodegradable. In addition, their prolonged use can generate resistance that reduces its effectiveness. For these reasons, alternative antimicrobial and virucidal agents of natural origin have been developing including carbohydrates, alcohols, esters, aldehydes, and ketones. These essential oils are responsible for the biological properties of aromatic and medicinal plants. Several essential oils and their components also have pharmacological effects due to their proven anti-inflammatory, antioxidant, and anticancer properties. However, the extraction of these essential oils involves a very high cost, since very large amounts of raw material are needed, and they have a very significant negative impact on the ecosystem from which they are obtained.

Although the antimicrobial and viricidal effect of some of the active ingredients of said essential oils has been previously demonstrated, the minimum amount of said active ingredients that are necessary to ensure an inhibitory activity, that is, what is known as minimum inhibitory concentration (MIC), are too high (Halldor Thormar, Lipids and Essential Oils as Antimicrobial Agents, Wiley Ed). It implies that the effective biocidal amount of these active ingredients is very high, and it has a direct and unaffordable impact in the cost of production of antimicrobial products that include them, in addition to the already mentioned negative effect on the environment. Furthermore, the higher the necessary dosage amount of these active ingredients, the greater the possibility of occurring adverse reactions to them.

Therefore, from what is known in the state of the art it is derived that there is still the need of providing effective, safety, stable, and cheap compositions having an immediate and prolonged broad-spectrum biocidal activity, without harmful the environment.

### SUMMARY OF THE INVENTION

The inventors have found that the specific composition of 5-isopropil-2-metilfenol, 1,3,3-trimethyl-2-oxabicyclo[2.2.2]octane, 4-methyl-1-(propan-2-yl)cyclohex-3-en-1-ol, and, optionally, 5-methyl-2-(propan-2-yl) phenol and/or 5-methyl-2-(propan-2-yl)cyclohexan-1-ol of the present invention is useful as biocide agent. In fact, the composition of the present invention has an enhanced a broad-spectrum biocidal activity. As it is demonstrated in the experimental section, the composition of the present invention has an unexpectedly effective activity as bactericidal, fungicidal, virucidal and yeasticidal, even when very low concentrations are used. In fact, this enhanced effect is much higher than the sum of biocide effect of each one of the active ingredients separately (as it is demonstrated in the examples below). Therefore, the composition of the present invention shows a synergistic biocide effect even at much lower concentrations than the ingredients separately. As it is demonstrated in the experimental section, this enhanced effect is shown in the treatment or prevention of infected surfaces such as living tissues (i.e. skin, mucosa, and soft tissues among others) and non-living/in-aminate objects/surfaces; and also for the treatment or prevention of space/environment for a domestic use or a professional activity. In fact, the effectivity of the compositions of the invention is higher and prolonged in time than the effectivity shown for bleach. Furthermore, as the ingredients forming part of the composition of the invention are non-toxics, the disadvantages associated to the use of bleach are overcome.

The composition of the present invention is also advantageous because is versatile being useful in the manufacturing of products for several fields such as pharmacy, veterinary, cosmetic, agro- or phytosanitary field, hygiene/cleaning industry and perfumery fields. In fact, the composition of also useful in the food/feed industry for human/animal consumption since all active ingredients of the composition of the invention can be of “food/feed grade”. Also, because it may be used at such a low concentration, it does not alter the formulations of cosmetic products or generate any type of adverse effect (irritation, allergies, etc.) on the skin.

Furthermore, the compositions of the present invention are stable under manufacturing, storage, and normal use conditions; and can be used in low amounts without compromising the biocidal activity, such that, for example, a biocidal efficacy of up to 98.7% is maintained in 10 days from the first application of the product. In fact, they have a long half-life reducing the number of applications (spacing the use) without hindering the biocidal effect. It implies that the cost of its use is considerable reduced, and that the useful life of the surface or object to be treated is increasing. It is specially advantageous wherein the composition is use in the cosmetic, food/feed industry and perfumery fields that allows simplifying the procedure protocols spacing the frequency of application without compromising the quality and lowering the economic cost. Besides, biocidal efficacy of the compositions is maintained throughout a broad pH range and achieve surprisingly good disinfection even when applied by fogging (shown in the examples below).

In addition, the composition of the present invention also show an unexpected synergic effect when combined with other biocidal active ingredient. As it is demonstrated in the experimental data, the combination of the composition of the present invention with other commonly used bactericidal and viricidal active ingredients allows increasing synergistically their activity and also widen the activity spectrum. This effect is commonly known as biocidal booster effect. Without being bound to any theory, this effect can be associated to the fact that the composition of the invention is also useful for inhibiting the adherence of the pathogens to the surface since inhibits the growth of biofilm. It means that apart from its inherent capacity of killing pathogens, the composition of the invention is also useful as adjuvant to other biocide active ingredients and as a biocide booster.

As it is demonstrated in the experimental section, the composition of the present invention is effective against the most common pathogens (including bacteria, fungi, and yeast) that cause a higher incidence of disease in human and animals. This high effectivity and broad spectrum is achieved even of using a low effective amount of the composition. Besides, the composition of the present invention is also effective against pathogens that have resistance to antibiotics (mainly because the spore formation capability-such as C. difficile) and new pathogens (SARS-Cov-2). The composition is also effective against biofilms formed by pathogenic microorganisms such as Pseudomonas aeruginosa or Acinetobacter Baumannii, thus being particularly useful is hospital environments where such biofilms and resistant pathogens are of great concern.

Finally, the compositions of the present invention have a very small impact on the environment and their use does not generate adverse effects on the ecosystem from which they are obtained or on living beings. The combinations further do not require rinsing after application for disinfection processes. The present inventors have carried out extensive research to arrive at the optimal combination of active ingredients that work synergically while maintaining a low toxicity profile.

Thus, the first aspect of the invention relates to a composition comprising:
(a) from 0.1% to 45% by weight of 5-Isopropil-2-metilfenol;
(b) from 0.1% to 45% by weight of 1,3,3-Trimethyl-2-oxabicyclo[2.2.2]octane;
(c) from 0.1% to 60% by weight of 4-Methyl-1-(propan-2-yl)cyclohex-3-en-1-ol;
(d) optionally, from 0.1% to 45% by weight of 5-Methyl-2-(propan-2-yl) phenol;
(e) optionally, from 0.1% to 45% by weight of 5-Methyl-2-(propan-2-yl)cyclohexan-1-ol,
(f) optionally, one or more additional active ingredients; and
(g) optionally, one or more appropriate acceptable excipients or carriers;
being the sum of ingredients up to 100% by weight.

The second aspect of the invention relates to a product comprising: (i) an effective amount of a composition as defined in the first aspect of the invention; and (ii) one or more appropriate acceptable excipients of carriers.

The third aspect of the invention is the use of the composition of the first aspect of the invention or the product of the second aspect of the invention in therapy (pharmacy and veterinary), cosmetic, agriculture, food/feed/dietetic industry, hygiene/cleaning industry, and scented/perfumery industry.

It is also part of the invention processes for the preparation of the compositions of the first aspect of the invention and the products comprising them of the second aspect of the invention.

### BRIEF DESCRIPTION OF DRAWINGS

FIG. 1 shows the antibacterial activity of sample 1 (1; composition comprising 1% (w/w) of the composition Example 22 of the present invention), sample 2 (2; composition comprising 2% (w/w) of the composition Example 22 of the present invention), versus no treatment (C1; negative control) and bleach (C2; as positive control) Test against vegetative cells of Clostridium difficile. The effectivity is shown as the variation of the optical density at 600 nm 48 h-0 h on the Y-axis (DO600/48 h-DO600/0 h) at the cited time (5, 15 and 30 min) for each one of the ribotypes 027 of Clostridium difficile. FIG. 1. A: C. difficile 027, 5 min contact; FIG. 1. B: C. difficile 027, 15 min contact; and FIG. 1. C: C. difficile 027, 30 min contact

FIG. 2 shows the antibacterial activity of sample 1 (1; composition comprising 1% (w/w) of the composition Example 22 of the present invention), sample 2 (2; composition comprising 2% (w/w) of the composition Example 22 of the present invention), versus no treatment (C1; negative control) and bleach (C2; as positive control) Test against vegetative cells of Clostridium difficile. The effectivity is shown as the variation of the optical density at 600 nm 48 h-0 h on the Y-axis (DO600/48 h-DO600/0 h) at the cited time (5, 15 and 30 min) for each one of the ribotypes 078 of Clostridium difficile. FIG. 2. A: C. difficile 078, 5 min contact; FIG. 2. B: C. difficile 078, 15 min contact; and FIG. 2. C: C. difficile 078, 30 min contact

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present invention, any ranges given include both the lower and the upper end-points of the range. Ranges given, such as weight, temperatures, times, weights, and the like, should be considered approximate, unless specifically stated.

The terms “percentage (%) by weight”, “weight/weight %” and “w/w %” have the same meaning and are used interchangeable. They refer to the percentage of each ingredient of the composition in relation to the total weight of the composition. For example, 1% by weight corresponds to 1 g in 100 g of total weight).

For the purpose of the present invention, the term “extract” refers to the conventional sense to refer to concentrated preparations of plants obtained by removing the active constituents from the plant with suitable means. Such actives constituents can be obtained from various parts of the plant using different extraction/distillation techniques. Suitable means for removal of the active ingredients include, for example, use of steam entrainment, CO₂, supercritical fluids, hydro distillation, microwaves, organic solvents depending on the plant of origin and the active ingredient/s it contains, being able to be performed in cold temperatures or any specific temperatures in each process for the adequate extraction. Active ingredients thus obtained are sometimes directly incorporated in food/feed, pharmaceutical or cosmetic compositions in a variety of forms, including a pure or semi-pure component, a solid or liquid extract, or a solid algae matter. Plant extracts contain not only one but multiple constituents, many of them active. Extracts comprising the active ingredients of the present invention (carvacrol, eucalyptol, terpinen-4-ol, thymol and/or menthol) may be obtained from plan extracts, for example, selected from the group consisting of Myrtaceae family extract (for instance genus eucalyptus extract such as Eucalyptus globulus; genus Melaleuca such as Melaleuca alternifolia); Lamiaceae family extract (for instance genus Mentha such as Mentha piperita; genus Thymus such as Thymus zygis, Thymus vulgaris, and Thymus capitata; and genus Origanum such as Origanum vulgare), Rutacea family extract (for instance genus Citrus extract such as Citrus reticulata), Cupressaceae family extract (for instance genus Juniperus such as Juniperus oxycedrus); and Pinaceae family extract (for instance genus Pinus such as Pinus sylvestris); and mixtures thereof. For the purpose of the invention the claimed amount corresponds to the pure active ingredients or such amount of extract that included the claimed amount of the pure active ingredient. For the purpose of the invention, the term “essential oil” refers to an extract which has been highly concentrated and purified. Therefore, the term “extract” encompasses “essential oils”. For the present invention, the active ingredients are preferably isolated or obtained by synthesis and used in “pure” form. The present ingredients are commercially available in pure form from several providers.

As it is mentioned above, the composition of the first aspect of the invention comprises: (a) from 0.1% to 45% by weight of 5-Isopropil-2-metilfenol; (b) from 0.1% to 45% by weight of 1,3,3-Trimethyl-2-oxabicyclo[2.2.2]octane; (c) from 0.1% to 60% by weight of 4-Methyl-1-(propan-2-yl)cyclohex-3-en-1-ol; (d) optionally from 0.1% to 45% by weight of 5-Methyl-2-(propan-2-yl) phenol; and (e) optionally from 0.1% to 45% by weight of 5-Methyl-2-(propan-2-yl)cyclohexan-1-ol; (f) optionally, one or more additional active ingredients; and (g) optionally, one or more appropriate acceptable excipients or carriers; being the sum of ingredients up to 100% by weight.

In the context of the invention, the compound (a) “5-Isopropil-2-metilfenol” and “carvacrol” have the same meaning and are used interchangeable. They refers to the compound having the CAS number 499-75-2 and the following structure:

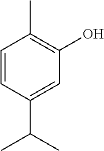

The composition of the invention comprises from 0.1% to 45% by weight of carvacrol. In an embodiment, the composition of the invention comprises from 0.5% to 45% by weight of carvacrol. In an embodiment, the composition of the invention comprises from 1% to 43% by weight of carvacrol. In an embodiment, the composition of the invention comprises from 2% to 43% by weight of carvacrol. In an embodiment, the composition of the invention comprises from 3% to 43% by weight of carvacrol. In an embodiment, the composition of the invention comprises from 3.5% to 40% by weight of carvacrol. In an embodiment, the composition of the invention comprises from 4% to 40% by weight of carvacrol. In an embodiment, the composition of the invention comprises from 30% to 40% by weight of carvacrol.

In an embodiment, the composition of the invention comprises from 15% to 40% by weight of carvacrol. In an embodiment, the composition of the invention comprises from 25% to 40% by weight of carvacrol. In an embodiment, the composition of the invention comprises from 30% to 40% by weight of carvacrol. In an embodiment, the composition of the invention comprises from 35% to 40% by weight of carvacrol. In an embodiment, the composition of the invention comprises from 25% to 43% by weight of carvacrol. In an embodiment, the composition of the invention comprises from 30% to 40% by weight of carvacrol. In an embodiment, the composition of the invention comprises from 30% to 35% by weight of carvacrol. In an embodiment, the composition of the invention comprises from 35% to 40% by weight of carvacrol.

In an embodiment, the composition of the invention comprises from 4% to 30% by weight of carvacrol. In an embodiment, the composition of the invention comprises from 4% to 20% by weight of carvacrol. In an embodiment, the composition of the invention comprises from 4.2% to 15% by weight of carvacrol. In an embodiment, the composition of the invention comprises from 4.5% to 10% by weight of carvacrol. In an embodiment, the composition of the invention comprises from 3% to 25% by weight of carvacrol. In an embodiment, the composition of the invention comprises from 3% to 12% by weight of carvacrol. In an embodiment, the composition of the invention comprises from 12% to 25% by weight of carvacrol.

In the context of the invention, the compound (b) “1,3,3-Trimethyl-2-oxabicyclo[2.2.2]octane”, “1,8-cineole”, and “eucalyptol” have the same meaning and are used interchangeable. They refers to the compound having the CAS number 470-82-6 and the following structure:

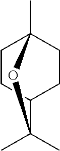

The composition of the invention comprises from 0.1% to 45% by weight of eucalyptol. In an embodiment, the composition of the invention comprises from 0.5% to 42% by weight of eucalyptol. In an embodiment, the composition of the invention comprises from 1% to 40% by weight of eucalyptol. In an embodiment, the composition of the invention comprises from 2% to 35% by weight of eucalyptol. In an embodiment, the composition of the invention comprises from 3% to 32% by weight of eucalyptol. In an embodiment, the composition of the invention comprises from 3.5% to 30% by weight of eucalyptol. In an embodiment, the composition of the invention comprises from 4% to 28% by weight of eucalyptol. In an embodiment, the composition of the invention comprises from 20% to 38% by weight of eucalyptol. In an embodiment, the composition of the invention comprises from 25% to 36% by weight of eucalyptol. In an embodiment, the composition of the invention comprises from 31% to 36% by weight of eucalyptol. In an embodiment, the composition of the invention comprises from 25% to 30% by weight of eucalyptol.

In an embodiment, the composition of the invention comprises from 3% to 30% by weight of eucalyptol. In an embodiment, the composition of the invention comprises from 3% to 12% by weight of eucalyptol. In an embodiment, the composition of the invention comprises from 12% to 30% by weight of eucalyptol.

In the context of the invention, the compound (c) “4-Methyl-1-(propan-2-yl)cyclohex-3-en-1-ol” and “terpinen-4-ol” have the same meaning and are used interchangeable. They refers to the compound having the following structure:

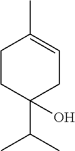

For the purpose of the present invention the term terpinen-4-ol encompasses both the enantiomers separately (i.e. R-enantiomer and S-enantiomer) and an “enantiomeric mixture” containing them. The term “enantiomeric mixture” as used herein refers to a mixture of the two enantiomers including the racemic mixture and any mixture enriched in one of the two enantiomers. The term “racemic mixture” or “racemate” as used herein refers to a mixture of both enantiomers which comprises equal amounts of the R-enantiomer and the S-enantiomer, which means that the molar ratio of the two enantiomers in the racemic mixture is 50:50. The term “mixture enriched in one enantiomer” as used herein refers to a mixture of enantiomers (R-enantiomer and S-enantiomer), which comprises a higher amount of one enantiomer with respect to the other enantiomer respectively, which means that the molar ratio of the two enantiomers in the enantiomerically enriched mixture is other than 50:50.

The term “enantiomeric excess” (ee) refers to the difference between the amounts of each of the enantiomers present in a mixture, relative to the total amount of the compound in the mixture expressed as percentage (×100%). For an excess of the R-enantiomer, the enantiomeric excess can be calculated by the following formula:

$$ee \% = ( [ R ] - [ S ] ) / ( [ R ] + [ S ] ) \times 100$$

For an excess of the S-enantiomer, the enantiomeric excess can be calculated by the following formula:

$$ee \% = ( [ S ] - [ R ] ) / ( [ R ] + [ S ] ) \times 100$$

The term “enantiomeric purity” (ep) refers to the purity of an enantiomer with respect to the other enantiomer. For an excess of the R-enantiomer, the enantiomeric purity can be calculated by the following formula:

$$ep \% = ( [ R ] / ( [ R ] + [ S ] ) \times 100$$

For an excess of the S-enantiomer, the enantiomeric purity can be calculated by the following formula:

$$ep \% = ( [ S ] / ( [ R ] + [ S ] ) \times 100$$

In the above formulas, [R] and [S] are the respective molar fractions of the enantiomers in a mixture such that [R]+[S]=1.

For the purpose of the present invention, the racemic mixture of the terpinene-4-ol refers to the compound of CAS number 562-74-3 having the following structure:

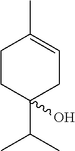

The (S)-enantiomer of the terpinene-4-ol refers to the compound of CAS number 2438-10-0 having the following structure:

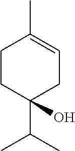

The (R)-enantiomer of the terpinene-4-ol refers to the compound of CAS number 20126-76-5 having the following structure.

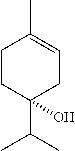

The composition of the invention comprises from 0.1% to 60% by weight of terpinen-4-ol. In an embodiment, the composition of the invention comprises from 10% to 57% by weight of terpinen-4-ol. In an embodiment, the composition of the invention comprises from 15% to 55% by weight of terpinen-4-ol. In an embodiment, the composition of the invention comprises from 17% to 54% by weight of terpinen-4-ol. In an embodiment, the composition of the invention comprises from 20% to 53% by weight of terpinen-4-ol. In an embodiment, the composition of the invention comprises from 22% to 52% by weight of terpinen-4-ol. In an embodiment, the composition of the invention comprises from 25% to 50% by weight of terpinen-4-ol. In an embodiment, the composition of the invention comprises from 21% to 38% by weight of terpinen-4-ol. In an embodiment, the composition of the invention comprises from 25% to 36% by weight of terpinen-4-ol. In an embodiment, the composition of the invention comprises from 31% to 36% by weight of terpinen-4-ol. In an embodiment, the composition of the invention comprises from 25% to 30% by weight of terpinen-4-ol.

In an embodiment, the composition of the invention comprises from 3% to 55% by weight of terpinen-4-ol. In an embodiment, the composition of the invention comprises from 3% to 12% by weight of terpinen-4-ol. In an embodiment, the composition of the invention comprises from 12% to 21% by weight of terpinen-4-ol. In an embodiment, the composition of the invention comprises from 21% to 55% by weight of terpinen-4-ol. In an embodiment, the composition of the invention comprises from 45% to 52% by weight of terpinen-4-ol.

In the context of the invention, the compound (c) “5-Methyl-2-(propan-2-yl) phenol” and “thymol” have the same meaning and are used interchangeable. They refers to the compound having the CAS number 89-83-8 and the following structure:

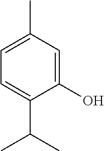

The composition of the invention comprises from 0.1% to 45% by weight of thymol. In an embodiment, the composition of the invention comprises from 1% to 42% by weight of thymol. In an embodiment, the composition of the invention comprises from 1% to 40% by weight of thymol. In an embodiment, the composition of the invention comprises from 1.5% to 38% by weight of thymol. In an embodiment, the composition of the invention comprises from 2% to 33% by weight of thymol. In an embodiment, the composition of the invention comprises from 2% to 32.5% by weight of thymol. In an embodiment, the composition of the invention comprises from 3% to 32% by weight of thymol. In an embodiment, the composition of the invention comprises from 1% to 10% by weight of thymol. In an embodiment, the composition of the invention comprises from 1% to 6% by weight of thymol. In an embodiment, the composition of the invention comprises from 3% to 6% by weight of thymol.

In an embodiment, the composition of the invention comprises from 0.01% to 35% by weight of thymol. In an embodiment, the composition of the invention comprises from 28% to 33% by weight of thymol. In an embodiment, the composition of the invention comprises from 0.01% to 1% by weight of thymol.

In the context of the invention, the compound (c) “5-Methyl-2-(propan-2-yl)cyclohexan-1-ol” and “menthol” have the same meaning and are used interchangeable. They refers to the compound having the CAS number 89-78-1 and the following structure:

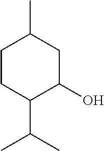

For the purpose of the present invention the term 5-Methyl-2-(propan-2-yl)cyclohexan-1-ol encompasses all possible isomers separately and also “isomeric mixtures” containing them including the racemic mixture.

One of the isomers of the 5-Methyl-2-(propan-2-yl)cyclohexan-1-ol is the compound having the CAS number 2216-51-5 and the following structure:

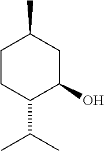

The composition of the invention comprises from 0.1% to 80% by weight of menthol. In an embodiment, the composition of the invention comprises from 0.5% to 40% by weight of menthol. In an embodiment, the composition of the invention comprises from 0.5% to 35% by weight of menthol. In an embodiment, the composition of the invention comprises from 1% to 30% by weight of menthol. In an embodiment, the composition of the invention comprises from 1.5% to 20% by weight of menthol. In an embodiment, the composition of the invention comprises from 2% to 15% by weight of menthol. In an embodiment, the composition of the invention comprises from 2.5% to 12% by weight of menthol. In an embodiment, the composition of the invention comprises from 1% to 10% by weight of menthol. In an embodiment, the composition of the invention comprises from 2% to 5% by weight of menthol. In an embodiment, the composition of the invention comprises from 3% to 4% by weight of menthol.

In an embodiment, the composition of the invention comprises from 5% to 35% by weight of menthol. In an embodiment, the composition of the invention comprises from 8% to 13% by weight of menthol. In an embodiment, the composition of the invention comprises from 27% to 32% by weight of menthol. In an embodiment, the composition of the invention comprises from 35% to 80% by weight of menthol. In an embodiment, the composition of the invention comprises from 60% to 80% by weight of menthol. In an embodiment, the composition of the invention comprises from 65% to 75% by weight of menthol.

The composition of the invention comprises (a) carvacrol, (b) eucalyptol and (c) terpinen-4-ol. In one embodiment, the composition consists essentially of (a) carvacrol, (b) eucalyptol and (c) terpinen-4-ol. In an embodiment, the composition comprises (a) carvacrol, (b) eucalyptol, (c) terpinen-4-ol, and (d) menthol. In an embodiment, the composition consists essentially of (a) carvacrol, (b) eucalyptol, (c) terpinen-4-ol, and (d) menthol. In an embodiment, the composition comprises (a) carvacrol, (b) eucalyptol, (c) terpinen-4-ol, (d) menthol, and (e) thymol. In an embodiment, the composition consists essentially of (a) carvacrol, (b) eucalyptol, (c) terpinen-4-ol, (d) menthol, and (e) thymol. In the sense of the present invention the expression “consisting essentially of” means that specific further components can be present, namely those not materially affecting the essential characteristics of the composition. The present invention also contemplates compositions as disclosed in the embodiments above or below, wherein the consisting essentially of is replaced by consisting of.

The ingredients can be provided from extracts containing them or included as pure or substantially pure substances, either isolated from an extract containing them or prepared synthetically. In all the embodiments above or below, the active ingredients are preferably included as pure ingredients (not as extracts comprising them). Using the pure ingredients has the advantage that the concentration of active ingredients in the composition may be tightly controlled, which is important to obtain the desired combination of active ingredients and to avoid other substances or contaminants which provide un-desired complexity or toxicity. In this sense, it is noted that extracts are often obtained using solvents that may be considered toxic.

In particular embodiments, the composition comprises or consists essentially of: (a) from 25% to 43% by weight of carvacrol; (b) from 20% to 38% by weight of eucalyptol; and (c) from 21% to 38% by weight of terpinen-4-ol; being the sum of ingredients up to 100% by weight. In particular embodiments, the composition further comprises or consists essentially of (d) from 0.1% to 9% by weight of thymol. In other particular embodiments, the composition further comprises or consists essentially of (d) from 1% to 9% by weight of menthol. In some embodiments, the composition further comprises or consists essentially of (f) one or more additional active ingredients and/or (g) one or more appropriate acceptable excipients or carriers. The sum of ingredients is always up to 100% by weight.

In a more particular embodiment, the composition comprises or consists essentially of: (a) from 30 to 40% by weight of carvacrol; (b) from 25 to 36% by weight of eucalyptol; (c) from 25 to 36% by weight of terpinen-4-ol; (d) optionally, from 1% to 6% by weight of thymol; and (e) optionally, from 2% to 5% by weight of menthol. In another more particular embodiment, the composition comprises or consists of (a) carvacrol from 30 to 35% by weight; (b) eucalyptol from 31 to 36% by weight; and (c) terpinen-4-ol from 31 to 36% by weight; and it does not contain thymol or menthol. In another more particular embodiment, the composition comprises or consists of (a) carvacrol from 35 to 40% by weight; (b) eucalyptol from 25 to 30% by weight; (c) terpinen-4-ol from 25 to 30% by weight; (e) menthol from 2 to 5% by weight; and it does not contain thymol. In another more particular embodiment, the composition comprises or consists of: (a) carvacrol from 35 to 40% by weight; (b) eucalyptol from 25 to 30% by weight; (c) terpinen-4-ol from 25 to 30% by weight; (d) thymol from 3 to 6% by weight; and (e) menthol from 2 to 5% by weight. In some embodiments, the composition further comprises or consists essentially of (f) one or more additional active ingredients and/or (g) one or more appropriate acceptable excipients or carriers. The sum of ingredients is always up to 100% by weight.

In particular embodiments, the composition comprises or consists essentially of: (a) from 3% to 25% by weight of carvacrol; (b) from 3% to 30% by weight of eucalyptol; and (c) from 3% to 55% by weight of terpinen-4-ol; being the sum of ingredients up to 100% by weight. In particular embodiments, the composition further comprises or consists essentially of (d) from 0.1% to 35% by weight of thymol. In other particular embodiments, the composition further comprises or consists essentially of (d) from 1% to 80% by weight of menthol. In some embodiments, the composition further comprises or consists essentially of (f) one or more additional active ingredients and/or (g) one or more appropriate acceptable excipients or carriers. The sum of ingredients is always up to 100% by weight.

In one embodiment, the composition comprises or consists essentially of: (a) from 5 to 12% by weight of carvacrol; (b) from 5 to 12% by weight of eucalyptol; (c) from 5 to 12% by weight of terpinen-4-ol; (d) optionally, from 0.1% to 6% by weight of thymol; and (e) from 50% to 80% by weight of menthol. In a particular embodiment, the composition comprises or consists essentially of: (a) from 7 to 10% by weight of carvacrol; (b) from 8 to 11% by weight of eucalyptol; (c) from 8 to 11% by weight of terpinen-4-ol; (d) optionally, from 0.1% to 3% by weight of thymol; and (e) from 65% to 75% by weight of menthol

In one embodiment, the composition comprises or consists essentially of: (a) from 2 to 8% by weight of carvacrol; (b) from 2 to 8% by weight of eucalyptol; (c) from 40 to 55% by weight of terpinen-4-ol; (d) from 25% to 35% by weight of thymol; and (e) from 5% to 15% by weight of menthol. In a particular embodiment, the composition comprises or consists essentially of: (a) from 3 to 6% by weight of carvacrol; (b) from 3 to 6% by weight of eucalyptol; (c) from 45 to 52% by weight of terpinen-4-ol; (d) from 27% to 33% by weight of thymol; and (e) from 7% to 12% by weight of menthol.

In a more particular embodiment, the composition comprises or consists essentially of: (a) from 15 to 35% by weight of carvacrol; (b) from 10 to 30% by weight of eucalyptol; (c) from 10 to 30% by weight of terpinen-4-ol; (d) from 0.01% to 5% by weight of thymol; and (e) from 20% to 35% by weight of menthol. In a more particular embodiment, the composition comprises or consists essentially of: (a) from 20 to 25% by weight of carvacrol; (b) from 21 to 28% by weight of eucalyptol; (c) from 21 to 28% by weight of terpinen-4-ol; (d) from 0.01% to 0.1% by weight of thymol; and (e) from 25% to 35% by weight of menthol.

In some particular embodiments the compositions of the above embodiments consist essentially of the disclosed ingredients. In other particular embodiments, the compositions of the above embodiments consist of the disclosed ingredients.

In an embodiment, the composition comprises or consists essentially of: (a) from 5% to 45% by weight of carvacrol; (b) from 0.5% to 42% by weight of eucalyptol; (c) from 10% to 57% by weight of terpinene-4-ol; (d) from 1% to 42% by weight of thymol; (e) from 0.5% to 40% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 1% to 43% by weight of carvacrol; (b) from 1% to 40% by weight of eucalyptol; (c) from 15% to 55% by weight of terpinene-4-ol; (d) from 1% to 40% by weight of thymol; (e) from 0.5% to 35% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 2% to 43% by weight of carvacrol; (b) from 2% to 35% by weight of eucalyptol; (c) from 17% to 54% by weight of terpinene-4-ol; (d) from 1.5% to 38% by weight of thymol; (e) from 1% to 30% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 3% to 43% by weight of carvacrol; (b) from 3% to 32% by weight of eucalyptol; (c) from 20% to 53% by weight of terpinene-4-ol; (d) from 2% to 33% by weight of thymol; (e) from 1.5% to 20% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 3.5% to 40% by weight of carvacrol; (b) from 3.5% to 30% by weight of eucalyptol; (c) from 22% to 52% by weight of terpinene-4-ol; (d) from 2% to 32.5% by weight of thymol; (e) from 2% to 15% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 4% to 40% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 15% to 40% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 25% to 40% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 30% to 40% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 35% to 40% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 4% to 30% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 4% to 20% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 4.2% to 15% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In an embodiment, the composition comprises or consists essentially of: (a) from 4.5% to 10% by weight of carvacrol; (b) from 4% to 28% by weight of eucalyptol; (c) from 25% to 50% by weight of terpinene-4-ol; (d) from 3% to 32% by weight of thymol; (e) from 2.5% to 12% by weight of menthol; (f) optionally, one or more additional active ingredients; and (g) optionally, one or more appropriate acceptable excipients or carriers; being the sum of ingredients up to 100% by weight.

In particular embodiments, the compositions disclosed in the embodiments above consist essentially of (a)-(c). In other particular embodiments the compositions disclosed in the embodiments above consist essentially of (a)-(d). In other particular embodiments the compositions disclosed in the embodiments above consist essentially of (a)-(e). In other particular embodiments the compositions disclosed in the embodiments above comprise (a)-(c) and optionally (d) and/or (e) as sole active ingredients, preferably as pure ingredients. In other particular embodiments the compositions disclosed in the embodiments above consist essentially of (a)-(g).

In an embodiment, the composition is one as disclosed herein below and above which has a pH equal to or lower than 8. In an embodiment, the composition is one as disclosed herein below and above which has a pH equal to or lower than 7.5. In an embodiment, the composition is one as disclosed herein below and above which has a pH equal to or lower than 7. These compositions are specially advantageous for the treatment or prevention of pathogen infection on living surfaces, for instance skin, scalp, and soft tissues.

In an embodiment, the composition is one as disclosed herein below and above which has a pH higher than 8. In an embodiment, the composition is one as disclosed herein below and above which has a pH higher than 9. These compositions are specially advantageous for the treatment or prevention of pathogen infection on inanimate surfaces and for water and air treatment/prevention.

In an embodiment, the composition of the invention further comprises one or more additional active ingredients. In an embodiment, the composition of the invention further comprises one or more additional active ingredients selected from the group consisting of antibiotic, viricide, fungicide, yeasticide, bactericide, skin conditioner, and prebiotic.

In an embodiment, the one or more additional active ingredients are selected from the group consisting of organic acid containing compound; quaternary ammonium containing compound, such as quaternary ammonium silane salt; methyl n-methyl anthranilate; guaiacol; p-cresol, cinnamaldehyde, geraniol, citral, methyl dihydrojasmonate (Hedione), 3-(3-propan-2-ylphenyl) butanal (florhydral), cis-para-menthanol (Cyclohexane methanol, 4-(1-methylethyl)-, cis-), delta-damascone, eugenol, p-cymene, alfa-pinene, beta-pinene, beta-caryophyllene, caryophylene oxyde, terpinene, and terpinolene. In particular, the one or more additional active ingredients are selected from the group consisting of lactic acid, citric acid, hyaluronic acid, pyruvic acid, ferulic acid, benzalkonium chloride, dimethyl octadecyl[3-(trimethoxy silyl)propyl]ammonium chloride, methyl n-methyl anthranilate; guaiacol; p-cresol, cinnamaldehyde, geraniol, and citral.

In an embodiment, the composition of the invention further comprises one or more quaternary ammonium containing compound; particularly selected from the group consisting of benzalkonium chloride, dimethyl octadecyl[3-(trimethoxy silyl)propyl]ammonium chloride, and mixture thereof; particularly from 0.01 to 50% by weight; and more particularly from 5 to 20% by weight.

In an embodiment, the composition of the invention further comprises a mixture of methyl n-methyl anthranilate; guaiacol; and p-cresol; as additional active ingredients; particularly, from 0.1% to 25% by weight of methyl n-methyl anthranilate; from 0.1% to 25% by weight of guaiacol; and from 0.1% to 25% by weight of p-cresol.

In an embodiment, the composition of the invention further comprises one or more additional active ingredients selected from the group consisting of cinnamaldehyde, geraniol, and citral; particularly from 5% to 20% by weight.

All the embodiments mentioned above for the compositions of the present invention comprising carvacrol, eucalyptol, terpinene-4-ol, and optionally thymol and/or menthol; also apply herein with the addition of one or more additional active ingredients mentioned above.

In an embodiment, one or more of the active ingredients of the compositions of the present invention can be in free form or alternatively in particulate form. In an embodiment, one or more of the active ingredients of the compositions of the present invention are in particulate form separately or in combination thereof selected from the group consisting of capsules (such as microcapsules or nanocapsules), and liposomes, micelles.

As mentioned above, the composition of the invention may further comprise one or more appropriate acceptable excipients or carriers. The appropriate excipients and/or carriers, and their amounts, can readily be determined by those skilled in the art according to the type of formulation being prepared and its application. In an embodiment, the one or more appropriate acceptable excipients or carriers are selected from the group consisting of humectant, surfactants, pH-regulating, gelling agent, solvents, co-solvents, rheological modifier agent, preservative, antioxidants, emulsifying agent, stabilizer, chelating agent, flavouring agent, masking, opacifier, binding agent, emollient, sweetener, fragrance, and perfume.

The term “pH-regulating” agent refers to acids or bases that can be used to adjust the pH of the finished product to the desired level, without affecting the stability of the solution. Examples of appropriate pH-regulating agents include, but are not limited to, acetic acid, lactic acid, citric acid, ethanolamine, formic acid, oxalic acid, potassium hydroxide, sodium hydroxide, triethanolamine, or their mixtures.

The term “gelling agent” refers to a compound that, when dissolved, suspended, or dispersed in a fluid (e.g., an aqueous fluid such as water or a buffer solution), forms a gelatinous semi-solid. Examples of gelling agents include but are not limited to hydroxyethyl cellulose, hydroxymethyl cellulose, hydroxypropyl guar, methyl cellulose, ethyl cellulose, hydroxypropyl cellulose, sodium carboxymethyl cellulose, carbomer, alginate, gelatin, gums (such as Arabic gum, guar gum, gellan gum and Xanthan gum) vegetal lecithin, and poloxamers.

The term “thickening agent” or “thickener” or “viscosity agent” or “rheology modifier agent” which is herein used interchangeably refers to a material that increases its viscosity without substantially modifying its other properties. Examples of appropriate viscosity agents include, but are not limited to, cellulose or their derivatives such as hydroxypropyl methylcellulose, polyethylene glycol, microcrystalline cellulose, cetearyl alcohol, alginates, branched polysaccharides, fumed silica, xanthan gum, carbomer, gums, gelatines, and polyacrylates.

The terms “solvent” and “co-solvent” refer to substances capable of dissolving or dispersing another substance or ingredient; particularly the active ingredients and/or excipients of the present invention. Examples of solvents include, but are not limited to water, alcohols such as ethanol, isopropanol, 3-methoxy-3-methyl-1-butanol; glycols such as butylene glycol, dipropylene glycol, dipropylene glycol methyl ether, and tripropylene glycol methyl ether; paraffin and isoparaffin waxes; silicone such as disiloxane and tetra siloxane; isopropyl myristate; (C₁₂-C₁₅) alkyl benzoate; and mixture thereof.

The term “preservative” refers to a material that prevents, reduces, or slows down microbial growth, providing that the stability of the solution is not affected. Examples of appropriate preservative agents include, but are not limited to, benzoic acid, butylparaben, ethylparaben, propylparaben, methylparaben, sorbic acid, potassium sorbate, sodium benzoate, phenoxyethanol, triclosan, or their mixtures.

The terms “filler” and “diluent” have the same meaning and are used interchangeably. They refer to any pharmaceutically acceptable excipient or carrier (material) that fill out the size of a composition, making it practical to produce and convenient for the consumer to use. Materials commonly used as filler include calcium carbonate, calcium phosphate, dibasic calcium phosphate, tribasic calcium sulfate, calcium carboxymethyl cellulose, cellulose, cellulose products such as microcrystalline cellulose and its salts, dextrin derivatives, dextrin, dextrose, fructose, lactitol, lactose, starches or modified starches, magnesium carbonate, magnesium oxide, maltitol, maltodextrins, maltose, mannitol, sorbitol, starch, sucrose, sugar, xylitol, erythritol and mixtures thereof. In an embodiment, the composition of the invention is one wherein the pharmaceutically acceptable excipients or carriers comprises one or more filler.

The term “emulsifying agent” or “emulsifier” which is herein used interchangeably refers to a material that reduces surface tension, promoting the formation of intimate mixtures of non-miscible liquids by altering the interfacial tension. Emulsifier stabilizes an emulsion by increasing its kinetic stability. Examples of appropriate emulsifier include, but are not limited to, glyceryl trioleate, glyceryl oleate, acetylated sucrose distearate, sorbitan trioleate, polyoxyethylene monostearate, glycerol monooleate, sucrose distearate, polyethylene glycol monostearate, octyl phenoxypoly (ethyleneoxy) ethanol, deacylerin penta-isostearate, sorbitan sesquioleate, hydroxylated lanolin, lecithin, lanolin, triglyceryl diisostearate, polyoxyethylene oleyl ether, calcium stearoyl-2-lactylate, sodium lauroyl lactylate, sodium stearoyl lactylate, cetearyl glucoside, methyl glucoside sesquistearate, sorbitan monopalmitate, methoxy polyethylene glycol-22/dodecyl glycol copolymer, polyethylene glycol-45/dodecyl glycol copolymer, polyethylene glycol 400 distearate and glyceryl stearate, candelilla/jojoba/rice bran polyglyceryl-3 esters, cetyl phosphate, potassium cetyl phosphate, or their mixtures

The term “stabilizer” refers to a material which lowers the surface tension of a liquid and the interfacial tension between two liquids, allowing their easier spreading. Surfactants have a hydrophilic head that is attracted to water molecules and a hydrophobic tail that repels water and simultaneously attaches itself to oil and grease in dirt. These opposing forces loosen the dirt and suspend it in the water, having the ability to remove it from surfaces such as the human skin, textiles, and other solids, when surfactants are dissolved in water. Examples of appropriate surfactant agents include, but are not limited to, non-ionic, ionic (either anionic or cationic) or zwitterionic (or amphoteric wherein the head of the surfactant contains two oppositely charged groups) surfactants. Examples of anionic surfactants include, but are not limited to, those based on sulfate, sulfonate or carboxylate anions such as perfluorooctanoate (PFOA or PFO), alkyl benzene sulfonate, soaps, fatty acid salts, or alkyl sulfate salts such as perfluorooctanesulfonate (PFOS), sodium dodecyl sulfate (SDS), ammonium lauryl sulfate, or sodium lauryl ether sulfate (SLES). Examples of cationic surfactants include, but are not limited to, those based on quaternary ammonium cations such as or alkyltrimethylammonium including cetyl trimethylammonium bromide (CTAB) a.k.a., or hexadecyl trimethyl ammonium bromide, cetylpyridinium chloride (CPC), polyethoxylated tallow amine (POEA), benzalkonium chloride (BAC), or benzethonium chloride (BZT). Examples of zwitterionic surfactants include, but are not limited to dodecyl betaine, cocamidopropyl betaine, or coco ampho glycinate. Examples of non-ionic surfactants include, but are not limited to, alkyl poly(ethylene oxide), alkylphenol poly(ethylene oxide), copolymers of poly(ethylene oxide), poly(propylene oxide) (commercially called Poloxamers or Poloxamines), alkyl polyglucosides including octyl glucoside and decyl maltoside, fatty alcohols including cetyl alcohol and oleyl alcohol, cocamide MEA, cocamide DEA, or polysorbates including tween 20, tween 80, or dodecyl dimethylamine oxide. Typically, the amount of surfactant in the composition of the invention can be from 0.01 to 20% by weight

The term “humectant” refers to a hygroscopic compound which attracts water molecules from the surrounding environment though either absorption or adsorption. Examples include but are not limited to, sodium hyaluronate, sodium hyaluronate, Glycerine, Liponic EG-1, Aquajuve, XIVIAC (xylitol), erythritol, Hyaluronic acid, Sorbitol, Collagen, and Aloe vera.

The term “emollient” agent refers to a compound that softens and soothes the skin in order to correct dryness and scaling of the skin, lubricating the skin surface, encouraging skin water retention, and altering product textures. Examples include but are not limited to, sodium hyaluronate, saecare DC, massocare CO, She Butter oil, Dimethicone, Lauric Acid, and Capric Triglyceride.

The term “masking” refers to a compound capable of weaking or disappearing the feeling of odor and/or bad taste. Examples include but are not limited to trisodium citrate (E331), arginine, sorbitol, and triethylene glycol.

The term “opacifier” refers to substances capable of reducing the transparent and translucent appearance of a cosmetic composition by increasing its opacity. Examples include but are not limited to Euperlan PCO, Lamesoft TM, and Tinovis AD.

The terms “binding agent” and “binder” have the same meaning and are used interchangeably. They refer to any pharmaceutically acceptable excipient or carrier (material) having binding properties. Examples include but are not limited to polyvinylpyrrolidone K30, methylcellulose polymers, hydroxyethyl cellulose, hydroxypropyl cellulose, L-hydroxypropyl cellulose (low substituted), hydroxypropylmethyl cellulose (HPMC), sodium carboxymethyl cellulose, carboxymethylene, carboxymethyl hydroxyethyl cellulose and other cellulose derivatives, starches or modified starches and mixture thereof.

The term “sweetener” refers to a compound that imparts a sweet flavour and usually provides no or very low energy. Examples include but are not limited to Erylite, sucralose, Stevia, Fructose, and Neohesperidin.

The term “chelating” agent refers to a compound that binds at multiple points in a coordination complex to a solubilized (metal) ion. Examples of appropriate chelating agents include, but are not limited to phytic acid, malic acid, nitrilotriacetic acid (NTA), ethylenediaminetetraacetic acid (EDTA), and alpha hydroxy acids

The term “flavouring” agent refers to a compound that supplements or strengthen the original flavour/taste of a substance. Examples of appropriate flavouring agents include, but are not limited to, sweetener, mint, strawberry, cherry, lemon, and liquorice, among others.

The term “fragrance” refers to a compound a combination of organic compounds that produces a distinct smell or odour. Examples of appropriate fragrances include, but are not limited to floral, fruity, woody, and musky, among others.

The term “perfume” refers to a liquid mixture of compounds used to emit a pleasant odour. The perfume is formed from fragrant essential oils derived from plants and spices and/or synthetic aromatic compounds. Examples of appropriate perfumes include, but are not limited to floral, fruity, woody, and musky, among others.

Another aspect of the invention is a process for the preparation of the compositions of the present invention as defined above. The compositions of the present invention can be prepared according to methods well known in the state of the art. The appropriate method and conditions can readily be determined by those skilled in the art according to the type of formulation being prepared.

The second aspect of the present invention refers to a product comprising:
(i) an effective amount of a composition as defined in the first aspect of the invention; and
(ii) one or more appropriate acceptable excipients of carriers.

The term “effective amount” refers to the amount of the composition of the present invention which provide the alleged technical effect, which is the broad-spectrum biocidal activity as disclosed herein above. The effective amount of the composition that corresponds to the appropriate amount of the composition of the invention in the product, will of course be determined by the skilled in the art regarding the particular circumstances surrounding the case, including the particular pathogen and the condition to be treated or prevent, and the surrounding considerations.

In an embodiment, the product of the present invention comprises from 0.001% to 50% w/w of the composition of the present invention. In an embodiment, the product of the present invention comprises from 0.001% to 7% w/w of the composition of the present invention. In an embodiment, the product of the present invention comprises from 0.001% to 5%. w/w of the composition of the present invention. In an embodiment, the product of the present invention comprises from 0.001% to 4% w/w of the composition of the present invention. In an embodiment, the product of the present invention comprises from 0.001% to 3% w/w of the composition of the present invention. In an embodiment, the product of the present invention comprises from 0.01% to 3% w/w of the composition of the present invention. In an embodiment, the product of the present invention comprises from 0.1% to 3% w/w of the composition of the present invention. In an embodiment, the product of the present invention comprises from 0.2% to 3% w/w of the composition of the present invention. In an embodiment, the product of the present invention comprises from 10% to 50% w/w of the composition of the present invention. In an embodiment, the product of the present invention comprises from 10% to 25% w/w of the composition of the present invention. In an embodiment, the product of the present invention comprises from 25% to 50% w/w of the composition of the present invention.

In an embodiment, the product of the present invention comprises from 0.1% to 2% w/w of the composition of the present invention. In an embodiment, the product of the present invention comprises from 0.1% to 1% w/w of the composition of the present invention. In an embodiment, the product of the present invention comprises from 0.1% to 0.5% w/w of the composition of the present invention, for example the product of the present invention comprises 0.2%, 0.3%, 0.4% w/w of the composition of the present invention. A product having a very high biocide activity with such low ingredient concentrations is only possible thanks to the synergic effect of the combination as disclosed in the present invention.

As it is demonstrated in the experimental section, the composition of the present invention is effective against the most common pathogens (including bacteria, fungi, and yeast) that cause a higher incidence of disease in human and animals. This high effectivity and broad spectrum is achieved even of using a low effective amount of the composition such as from 0.01 to 0.3% w/w. In fact, as it is demonstrated in the experimental section, the composition of the present invention is also effective against pathogens that have resistance to antibiotics (mainly because the spore formation capability). In this situation, the effective amount of the composition of the present invention can be from 0.1 to 5% w/w of the product. Nevertheless, these amount does not cause undesirable side effects (such as resistance or allergies) and it also has a very small impact on the environment and the ecosystem from which they are obtained or on living beings.

All the embodiments disclosed above for the composition of the first aspect of the invention including the active ingredients (a)-(e), additional active ingredients (f) and acceptable excipients and carriers also apply herein for the product of the second aspect of the invention.

The appropriate amount of the composition (i) as well as the excipients and/or carriers, and their amounts, can readily be determined by those skilled in the art according to the type of formulation being prepared and its application. Another aspect of the invention is a process for the preparation of the product of the present invention as defined above. The compositions of the present invention can be prepared according to methods well known in the state of the art. The appropriate method and conditions can readily be determined by those skilled in the art according to the type of formulation being prepared.

It is also a part of the invention, the composition of the first aspect of the invention or the product of the second aspect of the invention for use in therapy. In an embodiment, the composition of the first aspect of the invention; or alternatively, the product of the second aspect of the invention for use in therapy as biocide; particularly, for use in the treatment or prevention of human bacterial, viral, fungal and yeast disease or condition. Examples of bacterial, viral, fungal and yeast human disease or condition include, but are not limited to, VPH (Human Papilloma Virus), HBV (Hepatitis B), Coronavirus (including SARS-Cov-2), Herpes Virus, H7N9 (Influenza A), ECBO (Enterovirus Bovine), Rotavirus (viral Colitis), Vaccina Virus (smallpox), Polyoma Virus SV40, Bacteriophage for Lactobacillus, Poliovirus, Adenovirus, Norovirus (viral Colitis), Epstein-Barr, Polio virus type 1, LSc-2ab (Picornavirus), Adenovirus type 5, Strain Adenoid 75, ATCC VR-5, Murine norovirus, (Tobamovirus-ToBRFV, Strain S99 Berlin, Pseudomonas Aeruginosa, Escherichia Coli, Staphylococcus Aureus, Staphylococcus Epidermidis, Staphylococcus Hominis, Enterococcus Hirae, Burkholderia cepacia, Streptococcus mutans, Porphyromonas gingivalis, Fusobacterium nucleatum, Corynebacterium xerosis, Micrococcus spp, Lactobacillus acidophilus, Gardnerella vaginalis, Propionibacterium acnes, Legionella pneumophila, Proteus vulgaris, Salmonella (enteritis), Mycobacterium avium subsp. Paratuberculosis, Listeria monocytogenes, Fungal Spores (Teliospores, Zoospores, Ascospores, Zygospores), Bacillus, Clostridia, Xylella fastidiosa, Clostridium difficile, Erwinia amylovora, Xanthomonas arboricola pv. pruni; Candida albicans, Aspergillus brasiliensis, Aspergillus niger, Pityrosporum ovale, Campylobacter, Botrytis cinerea, Fusarium, Mildiu, Oidio, Phytophthora, Pythium, Fusarium oxysporum, Peronospora tabacina (tabaco), Phytophthora nicotinadiae (tabaco), Puccinia Hordei, Puccinia striiformis, Septoria tritici, Puccinia recondita, Puccinia graminis, Puccinia striiformis, Phytophthora cinnamoni Rands., Pythium spiculum, Pythium sterilum, Pythiaceae, Botryosphaeria corbicula, dotidiomycete fungus, ascomycete fungus, Order Xylariales, Biscogniauxia mediterranea, Agrobacterium tumefaciens, Pseudomonas savastanoi, and Agrobacterium vitis.

Therefore, the composition of the first aspect of the invention is a pharmaceutical composition and the appropriate acceptable excipients or carriers are pharmaceutically acceptable excipients or carriers; or alternatively, the product of the second aspect of the invention is a pharmaceutical product comprising: (i) a therapeutically effective amount of the composition; and one or more pharmaceutically acceptable excipients or carriers. The expression “therapeutically effective amount” as used herein, refers to the amount of active ingredients or composition containing them that, when administered, which is sufficient to prevent development of, or alleviate to some extent, one or more of the symptoms of a human disease or condition that involves a bacterial, viral, fungal and yeast infection. The particular dose of the active ingredients or composition containing them administered according to this invention will of course be determined by the skilled in the art regarding the particular circumstances surrounding the case, including the particular condition being treated, and the similar considerations. And the term “pharmaceutically acceptable excipients or carriers” refers to that excipient or carrier suitable for use in the pharmaceutical technology for preparing compositions with medical use.

In an embodiment, the composition of the first aspect of the invention or the product of the second aspect of the invention is useful for the prevention and/or treatment of skin/scalp/hair disease or condition that involves a disorder of the sebaceous gland. In an embodiment, the composition of the first aspect of the invention or the product of the second aspect of the invention is useful for the treatment or prevention of acne, rosacea, and dermatitis.

In an embodiment, the composition of the first aspect of the invention or the product of the second aspect of the invention is useful for the prevention and/or treatment of a disease or condition that involves an excessive or abnormal transpiration. Disease or conditions which involve an excessive or abnormal perspiration include hyperhidrosis, chromhidrosis and bromhidrosis.

It is also a part of the invention, the composition of the first aspect of the invention or the product of the second aspect of the invention for use in veterinary. In an embodiment, the composition of the first aspect of the invention; or alternatively, the product of the second aspect of the invention for use in veterinary as biocide; particularly, for use in the treatment or prevention of animal bacterial, viral, fungal and yeast disease or condition. Examples of bacterial, viral, fungal and yeast animal disease or condition include, but are not limited to, the porcine reproductive and respiratory syndrome (PRRS), Influenza A virus subtype H3N8, and Brucellosis. Therefore, the composition of the first aspect of the invention is a veterinary composition and the appropriate acceptable excipients or carriers are pharmaceutically acceptable excipients or carriers; or alternatively, the product of the second aspect of the invention is a pharmaceutical product comprising: (i) a veterinary effective amount of the composition; and one or more veterinary acceptable excipients or carriers. The expression “veterinary effective amount” as used herein, refers to the amount of active ingredients or composition containing them that, when administered, which is sufficient to prevent development of, or alleviate to some extent, one or more of the symptoms of an animal disease or condition that involves a bacterial, viral, fungal and yeast infection. The particular dose of the active ingredients or composition containing them administered according to this invention will of course be determined by the skilled in the art regarding the particular circumstances surrounding the case, including the particular condition being treated, and the similar considerations. And the term “veterinary acceptable excipients or carriers” refers to that excipient or carrier suitable for use in the veterinary technology for preparing compositions with veterinary use.

It is also a part of the invention, the use of the composition of the first aspect of the invention or the use of the product of the second aspect of the invention in cosmetic.

Therefore, the composition of the first aspect of the invention is a cosmetic composition and the appropriate acceptable excipients or carriers are cosmetically acceptable excipients or carriers; or alternatively, the product of the second aspect of the invention is a cosmetic product comprising: (i) a cosmetically effective amount of the composition; and one or more cosmetically acceptable excipients or carriers. The term used herein “cosmetically effective amount” refers to an amount enough to accomplish efficacies on cosmetic improvements in skin conditions described hereinabove. And the term “cosmetically acceptable” refers to that excipient or carrier suitable for use in contact with human skin without undue toxicity, incompatibility, instability, allergic response, among others for a non-medical use. The cosmetic composition of the present invention is designed to apply to the body to improve its appearance or to beautify, preserve, condition, cleanse, color or protect the skin, nails, or hair. Therefore, the above cosmetic compositions are adjectivally used for a non-medical application.

In an embodiment, the use of the composition of the first aspect of the invention or the use of the product of the second aspect of the invention in cosmetics is as a skin care agent. In an embodiment, the use of the composition of the first aspect of the invention or the use of the product of the second aspect of the invention in cosmetics is as a skin care agent wherein the skin care comprises ameliorating at least one of the following symptoms: roughness, flakiness, dehydration, tightness, chapping, and lack of elasticity.

In an embodiment, the use of the composition of the first aspect of the invention or the use of the product of the second aspect of the invention in cosmetics is as deodorant for reducing, minimizing, or eliminating the odour; particularly the body odour or clothes odour.

In an embodiment, the use of the composition of the first aspect of the invention or the use of the product of the second aspect of the invention in cosmetics is as antioxidant and/or preventive agent. It is shown that the composition of the invention delays the degradation/deterioration of the components of a product that contains it, in turn extending the useful life of the final product.

It is also a part of the invention, the use of the composition of the first aspect of the invention or the use of the product of the second aspect of the invention in agriculture. In an embodiment, the use of the composition of the first aspect of the invention or the use of the product of the second aspect of the invention in agriculture is as phytosanitary agent; particularly, for use in the treatment or prevention of plant bacterial, viral, fungal and yeast disease or condition. In an embodiment, the use of the composition of the first aspect of the invention or the use of the product of the second aspect of the invention in agriculture is as booster agent. The term “booster” refers to a compound capable of helping/upgrade the effectivity of other active ingredients, such as other biocide or fertilizers. For the purpose of the invention, the composition of the present invention is capable of boost the inherent biocide activity of other biocides, as it is demonstrated in the experimental section. Therefore, it is useful as co-adjuvant agent for use in the treatment or prevention of plant bacterial, viral, fungal and yeast disease or condition. Examples of bacterial, viral, fungal and yeast plant disease or condition include, but are not limited to plants and fruit trees such as fire blight, bacterial spot of stone fruit trees and almond trees. Therefore, the composition of the first aspect of the invention is a phytosanitary composition and the appropriate acceptable excipients or carriers are agriculturally acceptable excipients or carriers; or alternatively, the product of the second aspect of the invention is a phytosanitary product comprising: (i) a phytosanitary effective amount of the composition; and one or more agriculturally acceptable excipients or carriers. The expression “phytosanitary effective amount” as used herein, refers to the amount of active ingredients or composition containing them that, when administered, which is sufficient to prevent development of, or alleviate to some extent, one or more of the symptoms of a plant disease or condition that involves a plant bacterial, viral, fungal and yeast infection. The particular dose of the active ingredients or composition containing them administered according to this invention will of course be determined by the skilled in the art regarding the particular circumstances surrounding the case, including the particular condition being treated, and the similar considerations. And the term “agriculturally acceptable excipients or carriers” refers to that excipient or carrier suitable for use in agriculture for preparing compositions with phytosanity use. Examples of agriculturally acceptable excipients or carriers include, but not limited to, plant strengthens, nutrients, wetting agents, compounds that improve adherence, buffering compounds, stabilisers, antioxidants, osmotic protectors, and sunscreens.

In an embodiment, the composition of the first aspect of the invention is a dietary supplement and the appropriate acceptable excipients or carriers are edible acceptable excipients or carriers; or alternatively, the product of the second aspect of the invention is a dietary supplement comprising: (i) an effective amount of the composition; and one or more edible acceptable excipients or carriers. In terms of the present invention, the terms “dietary supplement”, “food/feed supplement” or “nutritional supplement” as used herein interchangeably refer to a composition or product intended to supplement the diet and provide nutrients, such as vitamins, minerals, fiber, fatty acids, or amino acids, which may be missing or may not be consumed in sufficient quantity in a person's or animal's diet. The term “edible” refers to compounds, compositions, complexes, salts, esters, excipients, and carriers that may be consumed by humans or animals without significant deleterious health consequences, it means that is suitable for consumption. For the purpose of the present invention the term “food/feed grade” and “edible” are equivalent and refers both the human and animal consumption. The particular dose of the active ingredients administered according to this invention will of course be determined by the skilled in the art regarding the particular circumstances surrounding the case.

The term “edible acceptable excipients or carriers” refers to excipients or carriers suitable for use in the preparation of compositions or products which can be consumed by humans or animals without significant deleterious health consequences.

It is also a part of the invention, the composition of the first aspect of the invention or the product of the second aspect of the invention for use in hygiene/disinfection/cleaning field.

In an embodiment, the composition of the first aspect of the invention; or alternatively, the product of the second aspect of the invention is useful as disinfectant. Therefore, the composition of the first aspect of the invention is a disinfectant composition and the appropriate acceptable excipients or carriers are acceptable excipients or carriers; or alternatively, the product of the second aspect of the invention is a disinfectant product comprising: (i) a disinfectant effective amount of the composition; and one or more acceptable excipients or carriers. The term “disinfectant” refers to a product which kills microorganisms on inanimate objects or spaces. Examples include hard surfaces, medical devices, surgical equipment, and air. The expression “disinfectant effective amount” as used herein, refers to the amount of active ingredients or composition containing them that, when administered, which is sufficient to kill bacteria, virus, fungi and yeast from inanimate surfaces or spaces. The particular dose of the active ingredients or composition containing them administered according to this invention will of course be determined by the skilled in the art regarding the particular circumstances. Suitable excipients or carriers for the disinfectant use can be anyone commonly used in antiseptic or disinfectant composition disclosed in the state of the art. For instance, the excipients or carriers can be selected from the group of dyes; flavours; solvents for instance glycols; diluents for instance water; surfactants for instance anionic surfactants; stabilizers; and rheologic modifiers. The composition of the present invention and the product containing it as disinfection include, but not limited to, disinfecting of utensils, equipment, facilities, air (environmental hygiene), surfaces and clothing; both at homecare services and professional care services. Examples include, disinfection of paediatric bottles and teats, facilities (toilet, kitchen, hotels, sanitary facilities-hospitals and adult or youth residences, sport centre, commercial centres, conference, and meetings rooms), vehicles (plain, bus, car, and motorcycle), and water treatment (wastewater treatment and potable water treatment). As it is mentioned above, the effectivity of the compositions of the invention is higher and prolonged in time than the effectivity shown for bleach.

It is also a part of the invention, the use of the composition of the first aspect of the invention or the use of the product of the second aspect of the invention in the food/feed industry. In an embodiment, the composition of the first aspect of the invention is a food/feed additive and the appropriate acceptable excipients or carriers are edible acceptable excipients or carriers; or alternatively, the product of the second aspect of the invention is a food/feed additive comprising: (i) an effective amount of the composition; and one or more edible acceptable excipients or carriers. In terms of the present invention, the terms “food/feed additive”, refer to a composition or product intended to maintain or improve the safety, freshness, taste, texture, or appearance of food/feed, which is added. The particular dose of the active ingredients administered according to this invention will of course be determined by the skilled in the art regarding the particular circumstances surrounding the case. In an embodiment, the use of the composition of the first aspect of the invention or the use of the product of the second aspect of the invention in the food/feed industry is selected from the group consisting of food/feed preservative, food/feed antioxidant, food/feed antiseptic, food/feed biocide, and technological adjuvant.

It is also a part of the invention, the use of the composition of the first aspect of the invention or the use of the product of the second aspect of the invention in the scented/perfumery sector. In an embodiment, the composition of the first aspect of the invention is a scented composition and the appropriate acceptable excipients or carriers are; or alternatively, the product of the second aspect of the invention is a scented product comprising: (i) an effective amount of the composition; and one or more acceptable excipients or carriers. The term “appropriate acceptable” for the scented compositions and products of the present invention refers to that excipients or carriers suitable for use in perfumery, particularly in the cosmetic and freshener field for the preparation of scented articles with perfumery use. The term “scented” refers to any article that is intended for consumers and contains the composition or the article of the present invention. In an embodiment, the scented article of the invention is in form of an air freshener. Examples of scented products of the present invention include air fresheners. The term “air freshener” refers to a scented article, which comprises the composition of the present invention and including the aroma/fragrance/perfume, which is appropriate for putting in contact with the air of a space, being the space a closed internal space such as for example rooms and cupboards; or an open space.

It is also a part of the invention, the use of the composition of the first aspect of the invention or the use of the product of the second aspect of the invention in apiculture. Bees are essential insects for planet earth. Unfortunately, they are very delicate insects which suffer greatly from pesticides, air and water pollution, deforestation, and many other environmental hazards derived from human activity. As a result, bee population has dramatically decreased over the last decades, threatening life in the planet itself. The inventors have found that the composition of the invention may be successfully used in apiculture. A product containing 0.2% of a composition according to the invention consisting of from 35 to 40% by weigh of carvacrol, from 25 to 30% by weight of eucalyptol, from 25 to 30% by weight of terpinen-4-ol, from 3 to 6% by weight of thymol, and from 2 to 5% by weight of menthol, was applied by nebulization to varroa infected beehives. The treatment successfully eliminated the parasite, without negatively affecting the bees. This is very surprising considering the fact that varroa mites have developed resistance to common biocides and, more importantly, that honeybees are so very sensitive to the biocides. Moreover, the bees were not repelled by the treatment, meaning that life and activity in the beehive continued as normal after the treatment. The composition of the invention, due to the its surprising efficacy and low toxicity achieved what hardly any other disinfectant has achieved in apiculture.

It is also a part of the invention, the use of the composition of the first aspect of the invention or the use of the product of the second aspect of the invention as a preservative in wood treatment, as an air disinfectant for both domestic and commercial use, either with electric devices or with the use of rechargeable or disposable batteries (single charge or with spare charges), as a bee hive disinfectant, as a bee feed supplement to eliminate pathogens such as, but not limited to, varroa, as a preservative and antioxidant in fuels, as a preservative in detergents and fabric softeners, as embalming and taxidermy liquids, as film preservatives (used for the preservation of films or coatings by controlling microbial deterioration or algal growth to protect the initial surface properties of materials or objects such as paints, plastics, sealants, wall adhesives, binders, papers or works of art), as protectors for fibres, leather, rubber and polymerised materials, as preservatives for building materials, as anti-mould products, as protectors for liquids used for working or cutting materials, or as products to control microbial spoilage of liquids used for working or cutting metals, glass or other materials.

The compositions and the products of the present invention can be formulated in several forms according to the route of administration/application, the type of galenic formulation and the field of the art intended to be used.

In an embodiment, the compositions or products can be in a galenic form selected from the group consisting of solutions, aerosols, non-aerosol sprays, sprays, shaving creams, powders, mousses, lotions, emulsions, gels, sticks, oils, ointments, pastes, creams, shampoos, shower gel, body washes, face washes, solid soaps, capsules, microcapsules, suspensions, liposomes, pellets, patch, and shower salts.

In an embodiment, the compositions or products are useful in the pharmaceutical, veterinary, cosmetic, and food/feed sectors and the route of administration is selected from the group consisting of oral, buccal, and topical.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### EXAMPLES

### A. General Consideration

Carvacrol, eucalyptol, terpinene-4-ol, thymol, menthol, methyl N-methyl anthranilate, guaiacol, p-cresol, dimethyl octadecyl[3-(trimethoxy silyl)propyl]ammonium chloride, benzalkonium chloride, and Polysorbate 20 (surfactant) were commercially available in food/feed grade by GUINAMA.

All strains of bacteria, fungi, and yeast are available from ATCC (https://www.lgcstandards-atcc.org/en/About/About_ATCC/Who_We_Are.aspx). All these strains were used as examples of bacteria, fungi, and yeast strains for performing the biocidal test of the compositions claimed in the present application. Other strains could also be used in their place to demonstrate the alleged biocide effect. Thus, the strains do not relate to the aim or focus of the invention.

### B. Minimal Compositions of the Present Invention

### 1. Minimum Inhibitory Concentration (MIC) Test

The objective of the test described in the following points is to determine the minimum inhibitory concentration (MIC) of the minimal composition (composition 1) and other compositions (compositions 2-3) of the present invention and compare them to the MIC of each of the ingredients (carvacrol, eucalyptol, terpinene-4-ol, thymol and menthol) taken on their own. compositions in table A were assayed against Pseudomonas aeruginosa microorganisms, and bacterial Biofilm. The compositions are shown in table A:

TABLE A
Compositions assayed for MIC. Concentration of the ingredients
in the compositions is expressed in % w/w

| | Carvacrol | Eucalyptol | Terpinene-4-ol | Thymol | Menthol |
|---|---|---|---|---|---|
| Composition 1 (Sanidermix 2.87) | 39% | 30% | 31% | | |
| Composition 2 (Sanisermix 2.23) | 38.7%a | 28.5% | 29.5% | 3.3% | |
| Composition 3 (Sanidermix pure) | 32% | 34% | 28.1% | 2.7% | 3.2% |
| Carvacrol only | 100% | | | | |
| Eucalyptol only | | 100% | | | |
| terpinene-4-ol only | | | 100% | | |
| Thymol only | | | | 100% | |
| Menthol only | | | | | 100% |

### Preparation of Compositions 1-3:

Step 1. Each ingredient was weight separately;
Step 2. Carvacrol, Terpinen-4-ol and eucalyptol were mixed together at low stirring.
Step 3 Then, the resulting mixture was heated to a temperature ranged from 40-50° C.
Step 4 (for compositions 2 and 3). After that, menthol was added, and the stirring was maintained to a temperature ranged from 60-80° C. until complete dissolution of menthol.
Step 5 (for composition 3). To the resulting mixture, solid thymol was added, and the resulting mixture was heated to a temperature ranged from 60-80° C. until thymol was dissolved;
Step 6. When a dissolution was obtained, the temperature was allowed to fall.

### Test Description:

An initial inoculation of the organisms of interest was carried out on the surface. After the inoculation, an initial sampling was carried out before beginning the process of applying the products on the different surfaces. To do this, surface samples were taken with a sterile swab. To take surface samples, a moistened swab was used, the swab was removed from its sterile packaging and the tip was moistened by immersing it in a tube containing the diluent/neutralizer. The tip of the swab was pressed against the sides of the tube to remove excess diluent/neutralizer. The tips of the swabs were placed on the surfaces to be examined and an estimated area of about 100 cm² was rubbed in each of the areas, while the handle of each of the swabs was rotated between the thumb and forefinger. The sampling was be carried out in vertical and horizontal directions, 10 times in each direction. Swabs were returned to each tube with diluent/neutralizer. Tubes were closed so that the swabs remained moist until analysis.

After the initial sampling, the application of the products was started on different surfaces at different concentrations. No need to rinse with water once the product was applied.

After the application, samples were taken from the different surfaces after 1 minute. The samples were taken following the sampling method described in the initial sampling (T=0).

All samples taken once the sampling process was finished were kept refrigerated for preservation until analysis.

Once the sampling process was finished, we proceeded to carry out the tests of the microorganisms of interest in the Laboratory. The assays were carried out by plating in a selective medium, following at all times the following procedure.

Starting from the initial solution, for each microorganism to be inoculated, a first bank of dilutions is made in TSB medium to obtain the sample at different concentrations. In this way, through turbidity, we can make a first screening and determine between which concentrations our CMI will oscillate. In this assay, the following initial dilutions are prepared for each strain: 1% (0.01 g/mL), 0.1% (1 mg/mL), 0.01% (0.1 mg/ml), 0.001% (0.01 mg/mL), 0.0001% (0.001 mg/mL).

All the tubes of the different concentrations are inoculated with each of the strains and incubated for 48 h at 37° C. To ensure that the technique used works correctly and obtain reliable results, we will also put:
Negative control sample: sample+TSB
Negative process control (C−): medium TSB
Positive control (C+): TSB medium+inoculum

With the results obtained in the first screening, make another bank of dilutions in TSB medium, delimiting between the concentration in which no growth is observed and the one in which bacterial growth is observed, so in this way we can adjust the MIC that we are looking for. We inoculated each tube with the different strains. This time we will sow each tube on a plate to be able to have a count and also observe what the MIC will be on the plate. We put it to incubate 48 h at 37° C. Once the incubation time has elapsed, read the tubes and plates and observe the minimum concentration in which there is no growth, this will be the Minimum Inhibitory Concentration.

Results obtained in tests against Pseudomonas aeruginosa (ATCC 15442) are shown in table B

TABLE B

| PRODUCTO | PARAMETER | MIC |
|---|---|---|
| Composition 1 | Minimum inhibitory concentration (MIC) | 0.16% |
| Composition 2 | Minimum inhibitory concentration (MIC) | 0.15% |
| Composition 3 | Minimum inhibitory concentration (MIC) | 0.09% |
| Eucalyptol | Minimum inhibitory concentration (MIC) | 1.09% |
| Carvacrol | Minimum inhibitory concentration (MIC) | 0.89% |
| Thymol | Minimum inhibitory concentration (MIC) | 2.11% |
| Terpinen-4-ol | Minimum inhibitory concentration (MIC) | 1.64% |
| Menthol | Minimum inhibitory concentration (MIC) | 5.32% |

Results obtained in tests against bacterial Biofilm (Listeria monocytogenes ATCC 35152, Aerobio 30° ATCC 25922, Clostridium sporogenes ATCC 19404, Candida albicans ATCC 26555 and Escherichia coli ATCC 10536) are shown in table C.

TABLE C

| PRODUCTO | PARAMETER | MIC |
|---|---|---|
| Composition 1 | Minimum inhibitory concentration (MIC) | 0.20% |
| Composition 2 | Minimum inhibitory concentration (MIC) | 0.19% |
| Composition 3 | Minimum inhibitory concentration (MIC) | 0.13% |
| Eucalyptol | Minimum inhibitory concentration (MIC) | 1.21% |
| Carvacrol | Minimum inhibitory concentration (MIC) | 1.01% |
| Thymol | Minimum inhibitory concentration (MIC) | 2.29% |
| Terpinen-4-ol | Minimum inhibitory concentration (MIC) | 1.96% |
| Menthol | Minimum inhibitory concentration (MIC) | 5.98% |

The above results shown in tables B and C show that the combination of Carvacrol, Terpinen-4-ol and

Eucalyptol has a MIC surprisingly lower that what would be expected from the MIC of each of the individual ingredients. Therefore, these results demonstrate that the minimal composition of the present invention has a synergistic effect between the 3 components that could not be expected. Adding Thymol and menthol to the minimal combination further improves the effectiveness of the minimal composition of the invention.

### 2. Biocidal Efficacy

The objective of the test described herein was to determine the biocidal action of the compositions 1, 2 as defined in the previous example, all used at 0.2% in water, against the microorganisms Pseudomonas aeruginosa (ATCC 15442), and Coronavirus (ATCC CCL-171).

### The Test Description:

An initial inoculation of the organisms of interest was carried out on the surface as described in section 1.1.

After the application, samples were taken from the different surfaces after 1 minute, 3 minutes and 7 minutes. The samples were taken following the sampling method described in the initial sampling (T=0).

All samples taken once the sampling process is finished were kept refrigerated for preservation until analysis. Once the sampling process was finished, we proceeded to carry out the tests of the microorganisms of interest in the Laboratory. The assays were carried out by plating in a selective medium, following UNE-EN 13697:2015.

| | | |
|---|---|---|
| Parameter | Composition 1 | Composition 2 |

Reduction after application of products (%) T1 min.

| | | |
|---|---|---|
| Pseudomonas aeruginosa count | Rlog 6.000006000006 | Rlog 5.999945999946 |
| Coronavirus | Rlog 6.000006000006 | Rlog 5.96853055869449 |

Reduction after application of products (%) T3 min.

| | | |
|---|---|---|
| Pseudomonas aeruginosa count | Rlog 6.000006000006 | Rlog 6.000006000006 |
| Coronavirus | Rlog 6.000006000006 | Rlog 6.000006000006 |

Reduction after application of products (%) T7 min.

| | | |
|---|---|---|
| Pseudomonas aeruginosa count | Rlog 6.000006000006 | Rlog 6.000006000006 |
| Coronavirus | Rlog 6.000006000006 | Rlog 6.000006000006 |

In view of the results obtained, it can be concluded that the three compositions under the described conditions have biocidal efficacy against Pseudomonas aeruginosa and Coronavirus starting after 1 minute of their application.

### C. Other Formulas (Examples 1-22)

### 1. Examples 1-22 Illustrate Further Compositions of the Present Invention, which Comprise the Combination of the Active Ingredients Carvacrol, Eucalyptol, terpinene-4-ol, Thymol, and Menthol

Table 1-4 disclose the quantitative formulations of the compositions of Examples 1-22, where the amount of the ingredients in food/feed grade or the essential oils containing them are expressed in % by weight of the ingredient in relation to the total weight of the composition.

TABLE 1

| Name | Ex. 1 | Ex. 2 | Ex. 3 | Ex. 4 | Ex. 5 |
|---|---|---|---|---|---|
| Carvacrol | 40 | 25 | 30 | 35 | 43 |
| eucalyptol | 25 | 35 | 30 | 34 | 20 |
| terpinene-4-ol | 26 | 38 | 30 | 23 | 33 |
| thymol | 6 | 1 | 8 | 3 | 2 |
| menthol | 3 | 1 | 2 | 5 | 2 |
| Pine essential oil | — | — | — | — | — |
| Total weight | 100 | 100 | 100 | 100 | 100 |

TABLE 2

| Name | Ex. 6 | Ex. 7 | Ex. 8 | Ex. 9 | Ex. 10 | Ex. 11 | Ex. 12 | Ex. 13 |
|---|---|---|---|---|---|---|---|---|
| Carvacrol | 30 | 35 | 39 | 43 | 24 | 28 | 32 | 35 |
| eucalyptol | 28 | 32 | 25 | 20 | 22 | 25 | 20 | 16 |
| terpinene-4-ol | 28 | 22 | 25 | 30 | 21 | 18 | 20 | 24 |
| thymol | 7 | 3 | 5 | 2 | 5 | 2 | 4 | 1.5 |
| menthol | 2 | 3 | 2 | 2 | 1 | 2 | 1.5 | 1.5 |
| Methyl N-methyl | 1 | 2 | 2 | 1 | — | — | — | — |
| anthranilate | | | | | | | | |
| guaiacol | 2 | 2 | 1 | 1 | — | — | — | — |
| p-cresol | 2 | 1 | 1 | 1 | — | — | — | — |
| Dimethyl octadecyl [3- | — | — | — | — | 7.4 | 6 | 4 | 4.4 |
| (trimethoxy silyl)propyl] | | | | | | | | |
| ammonium chloride | | | | | | | | |
| Benzalkonium chloride | — | — | — | — | 5 | 4 | 3 | 2 |
| (preservative) | | | | | | | | |
| Polysorbate 20 | — | — | — | — | 14.6 | 15 | 15.5 | 15.6 |
| (Surfactant) | | | | | | | | |
| Total weight | 100 | 100 | 100 | 100 | | | | |

TABLE 3

| Name | Ex. 14 | Ex. 15 | Ex. 16 | Ex. 18 | Ex. 19 | Ex. 20 | Ex. 21 | Ex. 22 |
|---|---|---|---|---|---|---|---|---|
| Carvacrol | 35 | 35 | 35 | 39 | 35 | 40 | 37 | 4.95 |
| eucalyptol | 20 | 20 | 20 | 27 | 30 | 28 | 30 | 4.95 |
| terpinene-4-ol | 22 | 22 | 22 | 26 | 25 | 24 | 24 | 49.5 |
| thymol | 5 | 5 | 5 | 5 | 7 | 5 | 6 | 30 |
| menthol | 3 | 3 | 3 | 3 | 3 | 3 | 3 | 10.6 |
| cinnamaldehyde | 15 | — | — | — | — | — | — | — |
| geraniol | — | 15 | — | — | — | — | — | — |
| citral | — | — | 15 | — | — | — | — | — |
| Total weight | 100 | 100 | 100 | — | — | — | — | — |

The process for the preparation of the composition of Examples 1-22 comprises mixing the ingredients disclosed for each example in Tables above in established orders with different temperatures until complete dissolution, let cool to room temperature before proceeding to packaging and conditioning.

### General Process for the Preparation of Examples 1-5, and 18-22

Step 1. Each ingredient was weight separately;
Step 2. Carvacrol, Terpinen-4-ol and eucalyptol) were mixed together at low stirring. To the resulting mixture, solid thymol was added;
Step 3. Then, the resulting mixture was heated to a temperature ranged from 60-80° C. until the thymol was dissolved;
Step 4. After that, the menthol, was added and the stirring was maintained to that temperature in a closed reactor until complete dissolution of menthol; and
Step 5. When a dissolution was obtained, the temperature was allowed to rise.

### General Process for the Preparation of Examples 6-9

The process comprises:
Steps 1 and 2 disclosed above were performed;
Step 6. The resulting mixture was heated at 50-60° C.; and guaiacol and N-Methyl Anthranilate were subsequently added until total dissolution;
Then, steps 3-4 disclosed above were performed;
Step 7. To the resulting mixture, p-cresol was added until complete dissolution; and Step 5 disclosed above was performed.

### General Process for the Preparation of Examples 10-13

The process comprises:
Steps 1-5 disclosed above were performed;
Step 7. To the resulting mixture polysorbate 20 was added under stirring at 50-60° C.;
Step 8. To the resulting mixture, benzalkonium chloride and subsequently Dimethyl octadecyl[3-(trimethoxy silyl)propyl]ammonium chloride was slowly added; and
Step 5 was finally performed.

### General Process for the Preparation of Examples 14-16

The process comprises:
Steps 1 and 2 disclosed above were performed;
Step 9. To the resulting mixture, cinnamaldehyde, geraniol and citral were subsequently and separately added at a temperature of 60-80° C.; and
Steps 3-5 disclosed above were finally performed.

### 2. Products of the Present Invention

Products of the present invention comprises concentration from 0.05% w/w to 5% w/w.

For example, for having a product with a concentration of 1% w/w of the composition of the invention, it is required 0.01 g of composition for 100 g of the total weight of the product.

For the purpose of the antibacterial test of the present invention, the process for the preparation of the products of the invention involves diluting the compositions of Examples 1-23 with deionized water and optionally one or more appropriate excipients or carriers, and one or more additional active ingredients until achieving the appropriate concentration expressed in weight/weight.

### 3. Comparative Examples Falling Outside the Scope of the Invention (Comparative Examples 1-4)

Comparative Examples 1-4 illustrate compositions, which do not comprise the combination of the active ingredient 5-Isopropil-2-metilfenol (carvacrol), 1,3,3-Trimethyl-2-oxabicyclo[2.2.2]octane (eucalyptol), 4-Methyl-1-(propan-2-yl)cyclohex-3-en-1-ol (terpinene-4-ol), 5-Methyl-2-(propan-2-yl) phenol (thymol), and 5-Methyl-2-(propan-2-yl)cyclohexan-1-ol (menthol) of the present invention.

In particular, the comparative compositions do not comply one or more of the ingredients or alternatively, comprise all ingredients but at least one of them in an amount falling outside the claimed range.

Table 5 discloses the quantitative formulations of the comparative compositions of Examples 1-4, where the amount of the ingredients in food/feed grade are expressed in % by weight of the ingredient in relation to the total weight of the composition.

TABLE 5

| Name | Comp. Ex. 1 | Comp. Ex. 2 | Comp. Ex. 3⁽¹⁾ | Comp. Ex. 4 |
|---|---|---|---|---|
| Carvacrol | 50 | 50 | — | 16 |
| eucalyptol | 15 | 50 | — | 50 |
| terpinene- | 25 | — | 75 | 26 |
| 4-ol | | | | |
| thymol | 7 | — | 12.5 | 5 |
| menthol | 3 | — | 12.5 | 3 |
| Total weight | 100 | 100 | 100 | 100 |

⁽¹⁾Corresponding to Example 4 of the European patent application EP0244363 without chlorhexidine

The process for the preparation of the comparative composition of Examples 1 and 4 is the general process for the preparation of the composition of Example 1 of the present invention as disclosed above, using the amount of each one of the ingredients specify in Table 5 above. For comparative composition Examples 2-3, the process is the general process for the preparation of the composition of Example 1 of the present invention as disclosed above but using Only the ingredients that are present.

### 4. Biocide Analytical Test

### 4.1. Antibacterial Analytical Test

The aim of the antibacterial analytical Test is determining the lowest concentration of the compositions of the present invention capable of inhibiting the growth of a given strain of bacteria.

The procedures adopted to prove the antimicrobial activity follow the European Standard protocols UNE-EN 1276:2010 standard for bactericidal testing in the medical area and surface disinfectant conditions.

The appropriate concentration of the compositions of the invention present in the product of the present invention capable of inhibiting the grown of the given strain of a bacterium is disclosed herein below.

The results for the compositions of Examples 1, 3, 4, 5, 11, 12, 13 and 14, complying with the standard were the followings:
Pseudomonas aeruginosa ATCC15442: 0.1% (w/w)
Staphylococcus aureus ATCC 6538:0.1% (w/w)
Staphylococcus epidermidis ATCC 12228:0.1% (w/w)
Staphylococcus hominis ATCC 27845:0.1% (w/w)
Enterococcus hirae ATCC 10541:0.1% (w/w)
Escherichia Coli ATCC 10536:0.1% (w/w)
Streptococcus mutans ATCC 25175:0.1% (w/w)
Porphyromonas gingivalis ATCC 53978:0.1% (w/w)
Fusobacterium nucleatum ATCC25586: 0.1% (w/w)
Corynebacterium xerosis ATCC7711: 0.1% (w/w)
Micrococcus spp ATCC 700405:0.1% (w/w)
Lactobacillus acidophilus ATCC 4356:0.1% (w/w)
Gardnerella vaginalis ATCC 14018:0.1% (w/w)
Propionibacterium acnes ATCC 11828:0.1% (w/w) 1 Burkholderia cepacian ATCC 17759:0.005% (w/w)

The results for the compositions of Examples 15 and 16, complying with the standard mentioned above were the followings:
Escherichia Coli ATCC 10536:0.1% (w/w)
Staphylococcus spp ATCC 27626:0.1% (w/w)

The result for the composition of Examples 17, complying with the standard mentioned above was the followings:
Escherichia Coli ATCC 10536:0.1% (w/w)

Therefore, these results demonstrated that the compositions of the present invention, even with very low concentrations from 0.005 and 0.1% (w/w) are used, are appropriate for inhibiting the growth of the most common bacterial pathogens.

Other study against Legionella pneumophila ATCC 33152, Listeria monocytogenes ATCC 35152, and spores from species of Bacillus, Clostridium, and fungi (Teliospores, Zoospores, Ascospores, and Zygospores) was performed. On one hand, the use of the composition of Example 1 at concentrations of 0.1%, 0.2% and 0.3% complying with the guidelines established in UNE-EN-13697 in dirty conditions allows having a concentration of Legionella pneumophila ATCC 33152 lower than 10-3 after 5 min. On the other hand, for the spores (of fungi, bacillus, and clostridia) and Listeria monocytogenes ATCC 35152, several surfaces of stainless steel were inoculated with the pathogens. After 5 minutes, the use of said dilutions of 0.1%, 0.2% and 0.3% (w/w) of the composition of Example 1 complying with the guidelines established in UNE-EN-13697:2015 allows inhibit the growing of all the pathogens. Therefore, the absence of the microorganisms was detected.

### 4.2. Antiviral Analytical Test

The antiviral activity of the compositions of the present invention were tested.

Method: The method is according to the UNE-EN 14476:2014+A1.

Tested compositions: Compositions of Examples 1, 3, 4, 5, 11, 12, 13 and 14.

### Results

The results show the concentration of the tested composition and the time required for the inhibition of the growth of the most important viral pathogens in clean or dirty conditions:

The results, complying with the method in clean conditions, were the following:

| | | |
|---|---|---|
| HPV (Human Papillomavirus) | 0.2% (w/w) | 5 min |
| ATCC VRMC-29 ™: | | |
| ECBO virus ATCC VR-37: | 0.2% (w/w) | 5 min |
| Rotavirus ATCC CRL-2378.1: | 0.1% (w/w) | 15 min |
| Vaccina virus ATCC VR-1549: | 0.1% (w/w) | 5 min |
| Polyoma virus SV 40 ATCC pUCSV40-B2E: | 0.1% (w/w) | 5 min |
| Poliovirus ATCC VR-166: | 0.1% (w/w) | 5 min |
| Adenovirus ATCC VR-1516: | 0.1% (w/w) | 5 min |
| Norovirus ATCC TIB-71: | 0.1% (w/w) | 5 min |
| Hepatitis B (HBV) ATCC VR-3232SD: | 0.1% (w/w) | 5 min |
| Herpesvirus ATCC CCL-81: | 0.1% (w/w) | 5 min |

The results, complying with the standard method in dirty conditions, were the following:

| | | |
|---|---|---|
| Influenza A (H7N9) ATCC VR-95: | 0.1% (w/w) | 15 min |
| Coronavirus ATCC CCL-171: | 0.1% (w/w) | 5 min |
| Poliovirus type 1 LSc-2ab | 0.1% (w/w) | 5 min |
| (Picornavirus) ATCC VR-649: | | |
| Adenovirus type 5 (strain: 75) | 0.1% (w/w) | 5 min |
| ATCC VR-5: | | |
| Murine Norovirus, strain | 0.1 (w/w) | 5 min |
| S99 Berlin ATCC VR-1937: | | |
| Modified Vaccinia Ankara | 0.1% (w/w) | 5 min |
| (MVA) virus ATCC VR-1508: | | |

These results demonstrated that the compositions of the present invention, even at low concentration about 0.1-0.2% (w/w), are appropriate for eliminating the most important viral pathogens.

### 4.3. Antifungal and Yeasticidal Analytical Test

The activity of the compositions of the present invention were tested.

### Test 1

Method: The methods were according to the UNE-EN 1276:2010 standard for Aspergillus brasiliensis, and the UNE-EN 1650:2008+A1 standard for the rest of the fungal and yeast.

Tested compositions: Compositions of Examples 1, 3, 4, 5, 11, 12, 13 and 14.

### Results

The results show the concentration of the tested composition and the time required for the inhibition of the growth of the fungi and/or yeast, complying with the corresponding standard method mentioned above:
Aspergillus brasiliensis ATCC 16404:0.07% (w/w)
Aspergillus niger ATCC 1015: 0.1% (w/w) 15 minutes+10 seconds
Pityrosporum ovale ATCC 44341:0.1% (w/w) 5 minutes
Candida albicans ATCC 10231:0.1% (w/w) 15 minutes+10 seconds

### Test 2

Method: The method was according to the UNE-EN-13697:2015

Tested compositions: Compositions of Examples 1, 3, 4, 5, 11, 12, 13 and 14.

### Results

The results show the concentration of the tested composition and the time required for the inhibition of the growth of the fungi and/or yeast, complying with the corresponding standard method mentioned above:

| | |
|---|---|
| Fusarium sp. ATCC 10 231 | 0.2% (w/w) 15 minutes |
| Phytophthora sp ATCC 3663 | 0.2% (w/w) 15 minutes |
| Pythium sp. ATCC 24619 | 0.2% (w/w) 15 minutes |

These results demonstrate that the compositions of the present invention are effective against fungi and yeast, even at concentrations as low as 0.007 to 0.1% (w/w).

### 4.4. Comparative Antibacterial Test

The comparative Antibiotic test of the composition Example 1 of the present invention, in comparison with the comparative Examples 1-3 were performed.

### Method

Following the standard UNE-EN 1276:2010, the different antibacterial activity was measured for the different tested compositions against Pseudomonas aeruginosa ATCC 15442, Staphylococcus aureus ATCC 6538, and Enterococcus hirae ATCC 10541.

### Results

MIC for the individual active ingredients is shown in table B And the concentration of the composition Ex. 1 and comparative Examples 1-3 capable of inhibiting the microorganism growth were measured against the tested microorganism:

| Microorganism | Ex. 1 | Comp. Ex. 1 | Comp. Ex. 2 | Comp. Ex. 3 |
|---|---|---|---|---|
| Pseudomonas aeruginosa ATCC 15442 | 0.1% >5 log | 1% 0 log | 0.5% >5 log | 1% 0 log |
| Staphylococcus aureus ATCC 6538 | 0.1% >5 log | 1% >5 log | 0.3% >5 log | 0.5% >5 log |
| Enterococcus hirae ATCC 10541 | 0.1% >5 log | 0.7% >5 log | 0.3% >5 log | 0.7% >5 log |

Therefore, as it is shown in Table above, only the composition of the present invention which comprises the specific combination of active ingredients has an effective antibacterial activity against the most important bacterial pathogens. In comparison, the concentration of the comparative composition Ex. 1 (which comprises carvacrol and eucalyptol in amounts falling outside the present invention) is much higher for all tested pathogens. On the other hand, these results all show that the combinations that do not comprise the main ingredients (carvacrol, terpinen-4-ol and eucalyptol)—see Comp. Ex. 3. containing eucalyptol, thymol, and menthol or Comp. Ex. 2. containing terpinen-4-ol and carvacrol-do not achieve such a surprising antibacterial effect. Therefore, these results demonstrate that the composition of the present invention has a synergistic effect between the 5 components at the claimed particular concentrations.

### 4.5. Conclusion

Therefore, the compositions of the present invention are effective as biocide for a wide spectrum due to its effectiveness for the elimination of pathogens such as bacteria, virus, fungi, and yeasts. The compositions of the present invention are specially advantageous because they maintain the biocide activity even at low concentration such as from 0.005% to 2% (w/w)

### 5. In Vivo Biocide Analytical Test

### 5.1. Antibacterial Activity Test in Wastewater

The antimicrobial capacity of the compositions of the present invention was evaluated in urban wastewater comprising a concentration of 10⁶ coliform bacteria per ml and 10⁴ faecal streptococci per ml. The wastewater was obtained from EDAR Emasesa y EDAR Aljarafesa.

### The Tested Compositions

The tested compositions were as follows:

Products of Examples 1, 3, 4, 5, 6, 7, 8, 9, 10 having a factor dilution of 20, 40, 50, 75 and 100 of the present invention, that is products 1.20-1.100; products 2.20-2.100; products 3.20-3.100; products 4.20-4.100; products 5.20-5.100; products 6.20-6.100; products 7.20-7.100; products 8.20-8.100; products 9.20-9.100; and products 10.20-10.100.

### Method

The method involved filtered 100 ml of said wastewater and adding of one of the products to be tested mentioned above of the present invention in a concentration and dilutions from 0.098% to 25% to evaluate the antibacterial activity against aerobic bacteria (i.e. Pseudomonas aeruginosa). The resulting mixture was maintained for 1 min at 22° C. with agitation and 2 days under incubation conditions.

### Results

All tested compositions at all tested concentration (even at the lowest tested concentration) result effective as biocide against the tested bacteria.

### 5.2. Antibacterial Activity Test in Environment

The air is a carrier of aerobic mesophilic microorganisms and their spores. Therefore, it can be the cause of contamination of food/feed and surfaces, as well as infections in humans and animals (skin and respiratory diseases). Mesophilic microorganisms include all bacteria, moulds, and yeasts capable of growing at 22° C. The count of said microorganisms serves to reflect the sanitary quality of the air and the handling conditions, being an indicator of contamination, without relating it to possible pathogens. In addition, certain germs that are not usually pathogenic if found in high amounts can lead to disease, so their determination provides great information on the effectiveness of treatments.

### Tested Compositions

The tested compositions were the composition of Example 1 at 0.2% (w/w).

### Method

In order to evaluate the antimicrobial capacity of the compositions of the present invention, nebulization tests were carried out with the tested compositions in a room of 65 m².

After nebulizing the tested composition of the invention, air samples (0.001 m³) were taken at 15 min, 30 min and 60 min. The samples were taken by filtration of 0.01 m³ of air and their subsequently impact on an agar plate by the use of apparatus VWR DUO SAS Super 360 sample. After that, the number of bacteria grown in the agar plate were counted and the subsequent measuring of the number of bacteria in the air was measured by plate colony count (UNE EN 1276 and UNE EN 1500)

### Results

The analysis of the agar plates showed that after 15 min of exposition of the compositions of the present invention, total absence of bacteria (aerobic), moulds and yeasts (0 cfu/m³) were detected.

Therefore, said analyses demonstrate that the compositions of the present invention are very effective in eliminating pathogenic elements present also in the environment.

### 5.3. Antibiotic Activity on Hands (Antiseptic Agent)

The antibacterial activity of the compositions of the present invention were also tested in hands by friction

Tested compositions: Compositions of Examples 1, 3, 4, 5, 11, 12, 13 and 14.

Method: according to UNE-EN 1500:2013 standard.

Results: the results show the concentration of the tested composition and the time required for the inhibition of the growth of bacteria in hands at a contact time of 45 seconds+5 seconds:
Escherichia coli ATCC 10536:0.1% (w/w)

### 5.4. Antibiotic Activity on Inert Surfaces (Disinfectant Agent)

The adhesion and subsequent growth of microorganisms on the surfaces of materials are a major concern in many biomedical applications, but also in the agri-food/feed industry and home hygiene.

### Short Term Evaluation

### Tested Compositions:

Dilutions from 0.0001% to 2% (w/w) of the compositions of Examples 1, 3, 4, 5, 11, 12, 13 and 14 were prepared.

### Method

To evaluate the ability of the compositions of the present invention to prevent the growth of microorganisms on inert surfaces, stainless steel, polyvinylchloride (PVC) and polyurethane (PU) discs were used, previously disinfected, and rinsed with distilled water.

The tested compositions mentioned above were used to impregnate the different discs. They were left to dry for 1 hour and then bacterial suspensions of Pseudomonas aeruginosa ATCC 15442, Staphylococcus aureus ATCC 6538, Escherichia Coli ATCC 10536, Candida albicans ATCC 10231 and Aspergillus brasiliensis ATCC 16404 were added separately. After 30 min of contact, the mixtures were spread in Petri dishes, and they were allowed to grow for 24-48 hours.

### Result

The concentration appropriate of the composition of the present invention to observe total absence of colonies on the plates on all the surfaces tested (that is stainless steel and plastic surfaces) are listed herein below.

### For the Compositions 11, 12, 13 and 14:

Pseudomonas aeruginosa ATCC 15442:0.05% (w/w)
Staphylococcus aureus ATCC 6538:0.05% (w/w)
Escherichia Coli ATCC 10536:0.05% (w/w)
Candida albicans ATCC 10231:0.05% (w/w)
Aspergillus brasiliensis ATCC 16404:0.05% (w/w)

### For the Compositions 11, 12, 13 and 14:

Pseudomonas aeruginosa ATCC 15442:0.1% (w/w)
Staphylococcus aureus ATCC 6538:0.1% (w/w)
Escherichia Coli ATCC 10536:0.1% (w/w)
Candida albicans ATCC 10231:0.1% (w/w)
Aspergillus brasiliensis ATCC 16404:0.1% (w/w)

### Long Term Evaluation

### Tested Compositions:

Dilutions from 0.0001% to 2% (w/w) of the compositions of Examples 11, 12, 13 and 14 were prepared.

### Method

The same method disclosed above were performed but the impregnated discs were left to dry for 15 days and then they were inoculated with the bacterial suspensions.

### Results

The results showed that the bactericidal effect of the tested compositions was not altered after 15 days.

These studies showed that the composition of the present invention is maintained even of being combined with other ingredients such as polymeric and non-polymeric cationic compounds, such as benzalkonium chloride and dimethyl octadecyl[3-(trimethoxy silyl)propyl]ammonium chloride.

5.5. Antibacterial Test of a Combination of the Composition of the Invention with Other Antibiotics. (Booster Antibiotic Capability)

The use of disinfectants with bactericide and virucide effects is very common for the treatment of surfaces or other common everyday objects. In the last few decades its presence in the market has become quite popular. This has led to the appearance of different substances or combinations with more or less effect against determined microorganisms. However, some of those biocides lose their effect after a few days, allowing the growth of microorganisms again.

The purpose of the present test is the evaluation of well-known homologated biocide in the treatment of a sterile container, with and without the complementary action of a composition of the present invention.

### Tested Composition:

Treatment A: Known biocide (lactic acid)
Treatment B: Composition of Example 1 of the present invention
Treatment C: known biocide (lactic acid)+Composition of Example 1 of the present invention

### Tested Bacteria⁽¹⁾

Pseudomonas aeruginosa ATCC 15 442,
Escherichia coli K12 CECT 433,
Staphylococcus aureus ATCC 6 538,
Enterococcus hirae ATCC 10 541

### Tested Virus⁽²⁾

Poliovirus ATCC VR-166,
Adenovirus ATCC VR-1516,
Norovirus ATCC TIB-71

### Method

Treatments A, B, and C were applied following the normalized work procedure PNT.08.93, and the differences between them were studied after 10 days of treatment on the sterile plastic container.

### Results

The tests were carried out on the different surfaces exposed to the mentioned bacteria and virus. Then, the bactericide and virucide activity of treatments A, B and C were measured. Subsequently, the measurement of the percentage of effectiveness of the tested composition on said microorganisms was performed. The percentage was calculated on the total of all the study microorganisms. The efficiency percentage obtained for each treatment is disclosed in Table below:

| Activity | Treatment A | Treatment B | Treatment C |
|---|---|---|---|
| Bactericide ⁽¹⁾ | 46.9% | 92.7 | 98.5% |
| Virucide ⁽²⁾ | 37.8% | 94.6 | 98.7% |

As it is shown in the Table above, a significant increase in both bactericide and virucide activity was observed when the biocide treatment is combined with the composition of the present invention. In fact, more than twice the effect compared to the biocide alone is observed and a result much closer to 100% compared to the composition of the present invention by itself.

Therefore, it can be concluded that the composition of the present invention has a synergistic effect in combination of known biocides, improving their biocide effect and also prolonging this effect in the time, achieving a long term biocide effect.

### 6. Pharmaceutical Products Comprising the Composition of the Present Invention

### 6.1. Products

The following products has been preparing using a composition of the present invention:

### Product 1

A sanitizing hand gel comprising 1% (w/w) of the composition Ex. 3:

| | | | | (%) |
|---|---|---|---|---|
| Phase | INCI name | commercial name | Function | by weight |
| Phase A | Pentylene glycol | Hydrolite-5 | Solvent | 3 |
| | Ethylhexylglycerin | Sensiva SC-50 | Skin Conditioner | 0.5 |
| | Propanediol | ZEMEA propanediol | Solvent | 5 |
| | Composition of Ex. 3 | — | Active ingredient | 1 |
| | Poloxamer 184 | Massocare PEL64 | Surfactant | 1 |
| | Polysorbate 20 | Tween 20 | Surfactant | 2.5 |
| Phase B | Distilled water | — | solvent | 86.2 |
| Phase C | Polyquaternium-10 | CELQUAT ® SC-230M | preservative | 4 |
| | | polymer | | |

| | |
|---|---|
| Total amount | 100 |

### Preparation Process:

Step 1. Preparation of phase A: Dispersing the components specified in table above in said order with agitation at room temperature
Step 2. Adding phase A obtained in previous step 1, to the water of phase B.
Step 3. Adding polyquaternium-10 (phase C), to the resulting mixture obtained in step 2 with slight stirring until the total dispersion of the polyquaternium-10 was achieved.

### Product 2

An intimate soap comprising 0.3% (w/w) of the composition Ex. 1

| Phase | ingredient | Function | % by weight |
|---|---|---|---|
| Phase A | Distilled water | Solvent | 39.37 |
| | Sodium hydroxide | pH regulator | 0.2 |
| | Sodium hyaluronate | Humectant | 0.1 |
| | Iscaguard PEG | Preservative | 1 |
| Phase B | Euperlan PCO | Opacifier | 2 |
| | Dermosooth/Bioecolia | Prebiotic | 0.5 |
| Phase C | Pureat glucol L | Surfactant | 18 |
| | Tegobetain F50 | Surfactant | 12 |
| Phase D | Composition of Ex. 1 | Active ingredient | 0.3 |
| | Lamesoft PO65 | Surfactant | 5 |
| Phase E | Distilled water | solvent | 20 |
| | citric acid monohydrate | pH regulator | 0.53 |
| | fine granular | | |
| | Trisodium citrate E331 | Masking | 1 |
| Total amount | | | 100 |
| pH | 4.0-4.5 | | |

### Preparation Process:

Step 1. Dissolve the components of phase A
Step 2. Dissolve the components of phase B in the water and incorporate them with agitation to phase A.
Step 3. Disperse the components of phase C and incorporate with stirring to mixture AB
Step 4. Add the components of phase D into the ABC mixture in order until the formation of a homogeneous paste without lumps.
Step 5. Incorporate with very gentle agitation phase E into ABCD mixture

### Product 3

A moisturizing intimate gels comprising 0.1%, 0.2% or 0.3% (w/w) of the composition Ex. 1.

| Phase | Ingredients | Function | % by weight |
|---|---|---|---|
| Phase A | Pentylene glycol | Solvent | 3 |
| | Ethylhexylglycerin | Skin Conditioner | 0.5 |
| | Vegetal Glycerine 99% | Humectant | 10 |
| | Liponic EG-1 | Humectant | 10 |
| | Hydroxyethyl cellulose | Binding Agent | 0.8 |
| | Xanthan Gum | Emulsifier | 0.2 |
| | Purified water | Solvent | 64.2 |
| Phase B | Polysorbate 20 | Surfactant | 1 |
| | Composition of Ex. 1 | Active ingredient | 0.3 |
| | Purified water | Solvent | 10 |

| | |
|---|---|
| Total amount | 100 |

### Preparation Process:

Step 1. Dissolve the components of phase A
Step 2. Dissolve the components of phase B in the water and add them with agitation to phase A.

### Product 4

A hand cream comprising 0.2% (w/w) of the composition Ex. 10:

| Phase | ingredients | Function | % by weight |
|---|---|---|---|
| Phase A | Distilled water | Solvent | 53.9 |
| | Vegetal Glycerine 99% | Humectant | 4 |
| Phase B | Stearic acid | Surfactant | 2 |
| | Glyceryl monostearate | Solvent | 1 |
| | Saecare DC | Emollient | 3 |
| | Massocare CO | Emollient | 5 |
| | Pemulen EZ-4U polymer | Emulsifier | 0.2 |
| Phase C | Amphisol K | Emulsifier | 0.3 |
| Phase D | Purified water | Solvent | 30 |
| | arginine | Masking | 0.1 |
| Phase E | Composition of Ex. 11 | Active ingredient | 0.5 |

| | |
|---|---|
| Total amount | 100 |

### Preparation Process:

Step 1. Dissolve the components of phase A
Step 2. Dissolve the components of phase B in the water and incorporate them with agitation to phase A.
Step 3. Dissolve the components of phase C and incorporate with stirring to mixture AB
Step 4. Dissolve the components of phase D and E into the water and incorporate them with very gentle agitation into ABC mixture.

### Product 5

A sanitizing hand cream comprising 0.2% (w/w) of the composition of Ex. 1.

| Phase | Ingredient | Function | % by weight |
|---|---|---|---|
| Phase A | Purified water | Solvent | 53.9 |
| | Vegetal Glycerine 99% | Humectant | 4 |
| Phase B | Stearic acid | Surfactant | 2 |
| | Glyceryl monostearate | Solvent | 1 |
| | Saecare DC | Emollient | 3 |
| | Massocare CO | Emollient | 5 |
| | Pemulen EZ-4U polymer | Emulsifier | 0.2 |
| Phase C | Amphisol K | Emulsifier | 0.3 |
| Phase D | Purified water | Solvent | 30 |
| | arginine | Masking | 0.1 |
| Phase E | Composition of Ex. 1 | Active ingredient | 0.5 |

| | |
|---|---|
| Total amount | 100 |

### Preparation Process:

Step 1. Dissolve glycerin into the water (phase A)
Step 2. Dissolve the components of phase B with agitation into phase A.
Step 3. Dissolve the components of phase C and incorporate with stirring to mixture AB

Step 4. Add the components of phase D into the ABC mixture in order until the formation of a homogeneous paste without lumps.
Step 5. Incorporate with very gentle agitation phase E into ABCD mixture

### Product 6

A vaginal gel comprising 0.3% (w/w) of the composition Ex. 5.

| Phase | ingredient | Function | % by weight |
|---|---|---|---|
| Phase A | Pentylene glycol | Solvent | 3 |
| | Ethylhexyl glycerine | Skin conditioner | 0.5 |
| | Glycerine vegetal 99% | Humectant | 5 |
| | Liponic EG-1 | Humectant | 5 |
| | Aquajuve | Humectant | 0.1 |
| | Hydroxyethylcellulose | Binding Agent | 0.8 |
| | Xanthan gum | Emulsifier | 0.2 |
| | Purified water | Solvent | 74 |
| Phase B | Polysorbate 20 | Surfactant | 1 |
| | Composition Ex. 5. | Active ingredient | 0.3 |
| | Lactic acid | Solvent | 0.1 |
| | Purified water | Solvent | 10 |

| | |
|---|---|
| Total amount | 100 |

### Preparation Process:

Step 1. Dissolve in water all components of phase A
Step 2. Dissolve the components of phase B in water with stirring and heat at 50° C.
Step 3. Incorporate phase B into phase A

### Product 7

A toothpaste comprising 0.2% (w/w) of the composition Ex. 13.

| Phase | ingredient | Function | % by weight |
|---|---|---|---|
| Phase A | Cosphaderm X34 | Emulsifier | 1 |
| | Glycerine vegetal | Solvent | 7 |
| | Sorbitol 70% | Masking | 15 |
| | Purified water | solvent | 7 |
| Phase B | Purified water | solvent | 41.85 |
| | Potassium nitrate | pH regulator | 1 |
| | XIVIAC (xylitol) | Humectant | 1 |
| | Erylite (Stevia 400) | Sweetener | 2 |
| | sucralose | Sweetener | 0.15 |
| Phase C | Iscaguard PEG | Preservative | 1 |
| | Massocare PEL64 | Surfactant | 1 |
| | Polysorbate 20 | Surfactant | 0.5 |
| | Peppermint essential | Flavouring | 0.2 |
| | oil 000125 | | |
| | Mint flavour 004354 | flavouring | 3 |
| | Mint flavour 84F021 | flavouring | 1 |
| | Mint flavour 84H890 | flavouring | 0.1 |
| | Composition Ex. 13 | Active ingredient | 0.2 |
| Phase D | Titanium dioxide E-171 | Opacifying | 1 |
| | Sorbitol AC39/AC36 | Masking | 5 |
| | Sorbitol TC15/TC10 | Masking | 7 |
| Phase E | AMPHO 2MA | surfactant | 4 |

| | |
|---|---|
| Total amount | 100 |

### Preparation Process:

Step 1. Disperse the Cosphaderm X34 in the glycerin and sorbitol and add finally the water with gentle agitation for its gelation
Step 2. Dissolve the components of phase B in the water and add them with agitation to phase A.
Step 3. Disperse the components of phase C and add with stirring to mixture AB
Step 4. add the components of phase D into the ABC mixture in order until the formation of a homogeneous paste without lumps.
Step 5. add with very gentle agitation phase E into ABCD mixture

### Product 8

A mouthwash comprising 0.2% (w/w) of the composition Ex. 14.

| | | | % by |
|---|---|---|---|
| Phase | ingredient | Function | weight |
| Phase A | Purified water | solvent | 68.19 |
| | Glycerine vegetal | solvent | 5 |
| | Erylite (stevia 400) | sweetener | 2 |
| | Potassium nitrate | pH corrector | 0.5 |
| | Sucralose KH-S60 | Sweetener | 0.1 |
| | XIVIA C (xylitol) | Humectant | 1 |
| Phase B | Purified water | Solvent | 12 |
| | Massocare PEL 64 | Surfactant | 4 |
| | (Poloxamer 184) | | |
| | LIPOCOL HC0-40 (PEG-40 | Emulgent | 5 |
| | hydrogenated castor oil) | | |
| Phase C | Mint flavour 004354 | Flavouring | 1 |
| | Composition Ex. 14 | Active ingredient | 0.2 |
| | (iscaguard PEG) | Preservative | 1 |
| | phenoxyethanol, triethylene) | | |
| Phase D | CI 74160 | Colorant | 0.01 |

| | |
|---|---|
| Total amount | 100 |

### Preparation Process:

Step 1. Dissolve the components of phase A
Step 2. Heat the components of phase B until transparent. Cool down at room temperature.
Step 3. Add phase C to phase B (when cold) and shake gently until formation of a practically transparent limpid solution. Then add phase BC to phase A. Stir until transparency.
Step 4. Add phase D and shake gently until a solutions forms clear transparent blue.

### Product 9

An oral spray comprising 0.2% (w/w) of the composition Ex. 14:

| Ingredients (INCI / CTFA) | Function | % by weight |
|---|---|---|
| Water | Solvent | 90.4 |
| Mint flavour 004354 | Flavouring agent | 2.5 |
| Erythritol | Humectant | 1.96 |
| Polysorbate 20 | Surfactant | 1.2 |
| Poloxamer 184 | Surfactant | 1 |
| Xylitol | Humectant | 1 |
| Phenoxyethanol | Preservative | 0.9 |
| PEG-40 Hydrogenated Castor Oil | Solvent | 0.5 |
| Triethylene Glycol | Masking | 0.1 |
| Sucralose | Sweetener | 0.1 |
| composition Ex. 14 | Active ingredient | 0.3 |
| Rebaudioside A | Skin conditioning | 0.02 |
| Limonene | Flavouring | 0.02 |

| | |
|---|---|
| Total amount | 100 |

### Preparation Process:

Step 1. Add into the water under stirring and slightly heating (40-45° C.) the components
Step 2. Leave under stirring until cooling to room temperature.

### Product 10

A gel comprising 0.2% (w/w) of the composition of Ex. 1.

| | ingredient | Function | % by weight |
|---|---|---|---|
| Phase A | Water | Solvent | 80 |
| | Sodium benzoate | Preservative | 3 |
| | EDTA | Chelating Agent | 2.5 |
| | Carbapol 940 | Emulsifier | 3 |
| Phase B | Clycerine Vegetal | solvent | 4 |
| | TEA | pH Corrector | 5 |
| Phase C | Eumulgin SML20 | Surfactant | 2 |
| | Composition Ex. 1 | Active ingredient | 0.2 |
| | Fragrance | Fragrance | 0.3 |

| | |
|---|---|
| Total amount | 100 |

### Preparation Process:

Step 1. Prepare of phase A: Dissolve Sodium Benzoate and EDTA together to carbapol 940 and dissolve the mixture in the water until total solubility. Leave undisturbed for 24 hour
Step 2. Add phase A the TEA and Glycerine. Stirring until total solubility. Phase AB
Step 3. Prepare phase C by mixing ingredients from phase C and add into phase AB.

### Product 11

A natural deodorant comprising 0.2% (w/w) of the composition of Ex. 5.

| Phase | ingredient | Function | % by weight |
|---|---|---|---|
| Phase A | Stearic Acid | Chelating Agent | 5 |
| | Propylene glycol | Surfactant | 46 |
| | Sodium Hydroxide | pH corrector | 1 |
| | Water | Solvent | 40 |
| Phase B | Alcohol | Solvent | 7 |
| | Composition Ex. 5. | Active ingredient | 0.3 |
| | Hexachlorophene | Deodorant | 0.1 |
| | Fragrance | Parfum | 0.2 |

| | |
|---|---|
| Total amount | 100 |

### Preparation Process:

Step 1. dissolve ingredients phase A with water
Step 2: dissolve active ingredient and hexachlorophene in alcohol. Add the fragrance, and finally mix phase B with phase A.

### Product 12

An antiperspirant blend comprising 0.2% (w/w) of the composition of Ex. 1.

| Phase | ingredient | Function | % by weight |
|---|---|---|---|
| Phase A | Aluminium Chlorhydroxide | Deodorant | 20 |
| | Propyleneglycol | Surfactant | 10 |
| Phase B | Purified water | Solvent | 40.4 |
| | Alcohol | Solvent | 25 |
| Phase C | Polysorbate | Surfactant | 4 |
| | Composition Ex. 1 | Active Ingredient | 0.3 |
| | Fragrance | Fragrance | 0.3 |

| | |
|---|---|
| Total amount | 100 |

### Preparation Process:

Step 1. Mix ingredients from phase A
Step 2. Add phase A into ingredients phase B. Phase AB
Step 3. Dissolve active ingredient and fragrance into polysorbate. Phase C Step 4. Mix phase AB with phase C under stirring.

### Product 13

A natural preservative blend for personal care comprising 0.2% (w/w) of the composition of Ex. 1.

| Phase | ingredient | Function | % by weight |
|---|---|---|---|
| Phase A | composition Ex. 1 | Active ingredient | 0.2 |
| | Polysorbate 20 | Surfactant | 0.4 |
| Phase B | Xanthan gum | Emulsifier | 0.8 |
| | Purified water | Solvent | 98.6 |

| | |
|---|---|
| Total amount | 100 |

### Preparation Process:

Step 1. Disperse composition ex. 1 in Polysorbate 20
Step 2. Add Xanthan gum
Step 3. Gradually add the water.

All products 1-13 of the present invention were prepared following the processes disclosed above and also following standard protocols for the manufacture of sanitary products y/o cosmetic products.

### 6.2. Stability, Toxicity, and Biocide Activity Tests

### A. Stability Test

### Products 7, 8 and 9

For three months the products 7, 8 and 9 were kept at different temperatures (5° C., 25° C. and 40° C.) while different physicochemical tests (i.e. appearance, color, odor, pH, and density) were carried out periodically every 30 days. Further, pH and density values were maintained over the course of 90 days, with a variance of less than 5% from the starting value for pH and less than 10% for density.

During the study, following the ISO standards, aerobic bacteria (ISO 21149:2017) and moulds and yeasts (ISO 16212:2017) were performed showing that the concentrations of pathogens were lower than 10 cfu/g. Meanwhile, Pseudomonas aeruginosa (ISO 22717:2016), Staphylococcus aureus (ISO 22718:2016), Escherichia coli (ISO 21150:2016) and Candida albicans (ISO 18416:2017) were completely absent, even after the 90 days.

Therefore, these results demonstrate that the compositions of the invention are stable under preparation conditions, storage conditions, and day-to-day common usage conditions since neither deterioration of the components nor loss of biocide activity were observed. Therefore, it was concluded that the products comprising the composition of the invention have a shelf life of 36 months

### B. Ocular Irritation Test

### Products 7, 8 and 9

Ocular irritation was also measured using the HET-CAM technique. The effect of oral products 7, 8 and 9 were evaluated on the chorioallantoic membrane (CAM) of a fertile egg and observed for 5 minutes. After that time, the resulting hyperaemia, haemorrhage, and coagulation were determinate assigning them values from 0 to 9 being 0 non-irritant and 9 irritant.

After performing the ocular irritant test, products 7-8 comprising the composition of the present invention non-irritation were observed obtaining a score of 0.

### C. Biocide Activity Test

### Viricidal Activity Test

Products 7-8 were submitted to biocide test according to UNE-EN 14476 for virus after a certain contact time. In particular, ECBO virus ATCC VR-37 (5 min), Rotavirus ATCC CRL-2378.1 (15 min), Vaccina virus ATCC VR-1549 (5 min), Polyoma virus SV40 ATCC pUCSV40-B2E (5 min), Bacteriophages for Lactobacillus ATCC 4356 (15 min), Poliovirus ATCC VR-166 (15 min), Adenovirus ATCC VR-1516 (5 min) and Norovirus ATCC TIB-71 (5 min) in clean conditions were studied. And Hepatitis B virus ATCC VR-3232SD (5 min), Coronavirus ATCC CCL-171 (5 min), Herpesvirus ATCC CCL-81 (HSV) (5 min), and Influenza A ATCC VR-95 (H7N9) (15 min) in dirty conditions were studied. In all cases, after the above given time, no presence of pathogen growing were observed. Thus, all tested products have a 100% of efficacy.
Product 3 were evaluated for its viricidal properties against HPV following the standard UNE-EN 14476:2014+A1. The results showed that the three concentrations of product 3 complied with this standard by establishing that only 5 min of contact was necessary to inhibit the growth of HPV and reduce its viability.

### Bactericidal Activity Test

Products 7-8 were submitted to biocide test according to UNE-EN 1500:2013 for bacteria after a certain contact time. In particular, Escherichia coli K12 ATCC 10536 (45 s) in clean conditions was studied; and for Candida albicans ATCC 10231 (5 min), Fusobacterium nucleatum ATCC 25586 (5 min), Porphyromonas gingivalis ATCC 53978 (5 min), Staphylococcus aureus ATCC 6538 (5 min), Lactobacillus casei ATCC 393 (5 min), Staphylococcus epidermis ATCC 12228 (5 min), and Streptococcus mutans ATCC 25175 (5 min) in dirty conditions was also studied.
Products 10-13 were evaluated for their antimicrobial properties at forced conditions against the pathogens disclosed in section 4.1. All the products were subjected to 41° C. for three months, after that time the analytical tests were carried out following standards UNE-EN 14476:2014+A1, UNE-EN 13697:2015, and UNE-EN 1500:2013. Further, products 11 and 13 were also submitted to the analytical tests following standards UNE-EN 1276:2010 and UNE-EN 1650:2008 to evaluate its activity against the pathogens disclosed in section 4.1. and also, their stability.

The obtained results shown that all the tested products comprising the composition of the present invention exhibit a broad-spectrum and effective activity in eliminating all tested pathogens, even at low concentration.

In fact, all the tested products were effective in the elimination of the most common pathogens evaluated under laboratory conditions. This effectivity was maintained even after being subjected to extreme external conditions, which mean that the compositions of the present invention are stable even under extreme storage conditions. Furthermore, the compositions also shown a high versatility for manufacturing different product in several galenic forms for different administration routes without compromising the stability, safety, and the biocide activity of the active ingredients.

### D. Antiseptic and Disinfectant Activity

### Product 1

Product 1 was submitted to biocide test according to UNE-EN 1500 for bacteria after a certain contact time In particular Escherichia coli K12 ATCC 10536 (45 s) in clean conditions was studied

### 7. Preservative Products Comprising the Composition of the Present Invention

The purpose of this study is the evaluation of the preservative activity in cosmetic products or edible products (food/feed sector) of the compositions of the present invention in comparison with the most commonly used (and commercially available) cosmetic preservatives.

### Tested Preservative

Preservative 1: product comprising the composition of Ex. 1 of the present invention

### Comparative Marketed Preservatives:

Comparative preservative 1: KATHON CG (Chloro-2-methyl-isothiazolin-36-one 2-methyl 4-isothiacolin-3-one)
Comparative preservative 2: ISCAGUARD PFA (Caprylyl glycol Phenethyl alcohol)
Comparative preservative 3: EUXYL K 701 (Phenoxyethanol Benzoic Acid; Dehydroacetic Acid and Ethylhexylglycerin)
Comparative preservative 4: ISCAGUARD DGP (Pentylene glycol; Caprylyl glycol and Decylene glycol)
Comparative preservative 5: ISCAGUARD PEG (Phenoxyethanol Triethylene glycol)
Comparative preservative 6: ISCAGUARD OP (Phenoxyethanol Caprylyl glycol)

### Method

The above mentioned commercial preservatives were tested along with the product of the present invention at doses from 0.1% to 1.5% against some of the most common bacteria, yeast, and fungus according to the Challenge Test according to ISO 11930:2012.

### Results

Table 6 shows the concentration (expressed in % (w/w) of the tested compositions that inhibit the pathogen growing.

TABLE 6

| | Preservatives | | | | | | |
|---|---|---|---|---|---|---|---|
| Pathogen | Ex. 1 | Comp. 1 | Comp. 2 | Comp. 3 | Comp. 4 | Comp. 5 | Comp. 6 |
| S. aureus | 0.1 | 0.15 | 0.2 | 0.5 | 0.8 | 0.47 | 0.4 |
| ATCC 6538 | | | | | | | |
| S. epidermis | 0.1 | 0.15 | — | 0.5 | — | — | — |
| ATCC 12228 | | | | | | | |
| E. coli | 0.1 | 0.3 | 0.1 | 0.25 | 0.8 | 0.44 | 0.2 |
| ATCC 10536 | | | | | | | |
| Candida albicans | 0.07 | 0.3 | 0.118 | 0.25 | 0.2 | 0.32 | 0.4 |
| ATCC 10231 | | | | | | | |
| P. aeruginosa | 0.1 | 0.3 | 0.1 | 0.5 | 0.8 | 0.22 | 0.4 |
| ATCC 15442 | | | | | | | |
| Aspergillus | 0.1 | 0.6 | 0.18 | 0.25 | 0.1 | 0.23 | 0.2 |
| niger/brasiliensis | | | | | | | |
| ATCC 1015/16 404 | | | | | | | |
| Burkholderia | 0.01 | 0.05 | — | 0.5 | — | — | — |
| cepacian | | | | | | | |
| ATCC 17759 | | | | | | | |

Further, the tested preservative of the invention was also tested against the following pathogens Staphylococcus hominis ATCC 27845, Enterococcus hirae ATCC 10541, Streptococcus mutans ATCC 25175, Porphyromonas gingivalis ATCC 53978, Fusobacterium nucleatum ATCC 25586, Corynebacterium xerosis ATCC 7711, Micrococcus spp ATCC 700405, Lactobacillus acidophilus ATCC 4356, Gardnerella vaginalis ATCC 14018, and Pityrosporum ovale ATCC 44341, according to the standard UNE-EN 1500:2013 and UNE-EN 14476: 2014+A1. The results shown that the composition of the present invention is also effective at a low concentration such as 0.1% against all the tested pathogens.

In conclusion, the above results show that the concentration of the composition of the present invention to inhibit the growth of the pathogen is much lower in comparison with any of the other products, and that it moreover covers a wider range of pathogens, making it a composition versatile and universal, being suitable for the preparation of products for different uses, regardless the route of administration and the galenic form prepared, without sacrificing effectiveness, stability, and safety.

### 8. Skin Irritation Clinical Assay

### 8.1. Cream Topical Application

The purpose of the present clinical test was to evaluate skin irritation, by measuring the skin irritability after the application of a topical cream product (Product 14) comprising Vaseline and 0.2% of the composition of Example 1 of the present invention.

To achieve that, 10 healthy volunteers of ages from 18 to 65 years old was included in the assay; 5 of the volunteers suffering from sensitive skin and the other 5 had normal skin.

The method involved the application of the Product 14 only on the right hand for 3 consecutive days. Each day, 30 minutes after the application of Product 14 of the invention, the skin was evaluated by a dermatologist to indicate any possible irritant responses in any of the patients.

None of the patients reported any adverse effect.

### 8.2. Topical Application of a Patch

The purpose of the present clinical test was to evaluate skin irritation, by measuring the skin irritability after the application of a product in form of a patch comprising the composition of the present invention. To achieve that, 10 healthy volunteers of ages from 18 to 65 years old was included in the assay.

The method involved applying a skin patch (Curatest® non-woven adhesive semi-occlusive patch made of polypropylene and hypoallergenic polyacrylate) with a surface of 50 mm² and 20 μl of the product comprising 0.2% (w/w) of the composition Example 1 of the present invention, that remained untouched on the skin for 48 hours. At the same time, a second comparative patch without tested product was placed, to use as control for any possible reaction caused by the patch itself.

After 48 hours, the patches were removed, and the experimental area was evaluated 30 minutes later to discard skin irritation produced by the patch. In addition, a re-evaluation was performed 48 hours after the removal of the patch in order to discard delayed signs of irritation.

The main common irritation signs (adverse effects) associate to the use of skin patch are erythema, edema, papules/vesicles/bullae/pustules, dryness/desquamation, and detergent effect (presence of wrinkles or worn aspect of the application area).

After 48 h and 96 h of the tested skin patch comprising the composition of the present invention, none of the previous irritation signs were observed. Thus, assessing the good compatibility of the product of the invention with the skin.

In view of both clinical studies, it is concluded that the composition of the present invention and the product comprising has shown very good skin compatibility, showing no irritation signs or any adverse effects.

### 9. Biofilm Formation Inhibition Test

The aim of this test is the assessment of the capability of the compositions of the present invention to inhibit the formation of biofilms apart from their biocide activity. Biofilm is a complex matrix consisting of extracellular polysaccharides, DNA, and proteins that protect bacteria from a variety of physical, chemical, and biological stresses allowing them to survive in hostile environments. Biofilm formation requires three different stages: cell attachment to a solid substrate, adhesion, and growth. The inhibition of one of these steps and/or react on specific targets (such as proteins) of the biofilm to leave pathogens armless against classical antibiotics and also preventing the onset of bacterial resistance and a slight effect on cell survival.

### Samples

Sample 1: comprising 0.2% (w/w) of the composition of Example 1
Sample 2: comprising 1% (w/w) of the composition of Example 1
Sample 3: comprising 2% (w/w) of the composition of Example 1
Sample 4: comprising 0.2% (w/w) of the composition of Example 1+0.1% lactic acid
Sample 5: comprising 2% (w/w) of the composition Example 22
Blank: no treatment

### Pathogens

2 strains of Pseudomonas aeruginosa isolated from patients (i.e. PA14, and PA14M)
2 strains of Pseudomonas aeruginosa from laboratory (i.e. PAO y PAOMUT).
PA14M and PAOMUT are colistin-resistant strains.

For the purpose of the present invention, other strains of Pseudomonas aeruginosa could also be used in their place to demonstrate the alleged effect. For example, Pseudomonas aeruginosa ATCC 15442, or Pseudomonas aeruginosa ATCC27853 which is a colistin-resistant strain.

### Method

A single colony was inoculated into 10 ml of Luria-Bertani (LB) broth, and incubated under agitation for 16 hours at 37° C. This culture was diluted in fresh Mueller-Hinton (MH) broth until achieving a concentration of 106 CFU/ml. Then, 100 μl of the resulting culture were put into flat-bottom 96-well microtiter or microtiter plates (Nunc TSP, System, Nunc, Roskilde, Denmark). These plates were covered with a lid having 96 polystyrene spikes around which the biofilm can develop (Nunc-TS, System, Nunc, Roskilde, Denmark). This system was incubated under favourable conditions for the biofilm formation, that was 48 h at 37° C. The spikes were washed with sterile water three times. They were placed back in another microtiter plate with 100 μl of fresh MH broth and the composition of the invention to be tested in each well. The final inoculum that adhered to the spikes was 4×107-2×108 CFU/ml.

These plates were incubated for 24 hours at 37° C. and the spikes were again washed three times, then placed in another plate with 100 μl of fresh MH broth in each well. The entire device was centrifuged for 10 min at 800 g to deposit the biofilm adhered to the spiker in the well.

After centrifugation (zero time), the optical density (OD) of the culture was measured by spectrophotometry

(Flow Titertek Multiskan Plus, Flor Laboratorios, Finland) at a wavelength of 595 nm. It was then incubated for 6 hours at 37° C. and the OD was measured again. Differences in OD values at time 0 and after 6 hours of incubation were determined, defining the minimum inhibitory concentration in biofilm as the first concentration of the composition in which no growth was observed.

Wells to which only culture medium was added were used as a negative control of sterility and as a positive control of growth in the absence of compound.

The experiment was revealed after incubating the microtiter plate for 6 hours, the optical density that indicated the growth in biofilm and the CMI-Biofilm (CMI-B) values were measured.

### Results

The biocidal effect of the tested samples of the present invention on the P. aeruginosa strains mentioned above after 15 min and 30 min of exposure at 25° C. and 37° C. versus control (no treatment) were measured. For PA14 strain: beneficial effect against the formation of biofilm were observed. And for PA14M, PAO and PAOMUT strains: inhibitory effects of the growth of the biofilm from 96% and 100% was observed in all tested conditions and also all tested compositions of the present invention.

The average biofilm growth inhibition (in percentage with 2 replicates for each strain) after 15 min and after 60 min of exposure, at 25° C. and 37° C., of the clinical strains PA14M, PAO and PAOMUT, compared to the untreated control was disclosed in Table below:

| | Average biofilm growth inhibition (%) | | |
|---|---|---|---|
| Sample | PA14M | PAO | PAOMUT |
| 1 | 96 | 96 | 99 |
| 2 | 100 | 99 | 97 |
| 3 | 100 | 100 | 100 |
| 4 | 100 | 100 | 100 |
| 5 | 100 | 100 | 100 |

Therefore, regarding the experimental data mentioned above, it can be concluded that the compositions of the present invention are capable of inhibiting the biofilm formation and its growth. It is specially advantageous because the compositions of the present invention have a dual effect: a direct biocidal effect and an inhibition of biofilm formation effect.

### 10. Antibacterial Activity Test Against Vegetative Cells of Clostridium difficile

Clostridioides difficile (C. difficile) is an anaerobic microorganism considered the main cause of diarrhoea associated with the use of antibiotics. This agent is responsible for a spectrum of diseases called C. difficile infection (CDI) ranging from uncomplicated diarrhoea to patient death. Although community transmission of this pathogen has been described, it mainly occurs in the hospital environment through spores. It is for this reason that patients with CDI are isolated to reduce its spread. Although the shapes Vegetative species of this pathogen die easily in the environment, it is capable of forming spores that allow them to survive for long periods of time (months and even years) until conditions are conducive to germination. The presence of spores has been detected in patient rooms and even on the gloves and uniforms of healthcare personnel who care for them.

The aim of the present test is the evaluation of the topical efficacy of the compositions of the present invention to reduce the transmission of C. difficile.

### Samples

Sample 1 comprising 1% (w/w) of the composition Example 22 of the present invention
Sample 2 comprising 2% (w/w) of the composition Example 22 of the present invention
Positive Control (C2): bleach at 1%
Negative Control (C1): untreated C. difficile 027/078

### Pathogens

Clostridium difficile belonging to ribotypes 027 y 078

In this study, C. difficile belonging to ribotypes 027 and 078 isolated from patients were used. Both strains were grown on blood agar plates (BioMerieuxR, Spain) and incubated for 24 h at 37° C. in anaerobiosis. For the purpose of the present invention, other ribotypes of Clostridium difficile could also be used in their place to demonstrate the alleged effect. For example, Clostridium difficile ATCC9689.

### Method

The isolated colonies of C. difficile 027 and C. difficile 078, grown on blood agar (BioMerieuxR, Spain), were inoculated in 10 mL of Brain Hearth Infusion Broth (Condolab R, Spain) until reaching a turbidity corresponding to pattern 3 of the MacFarlan scale (9×108 CFU/mL). These cultures were incubated for 8 h at 37° C. and under anaerobic conditions. This ensured that the cultures were in an exponential growth phase and, therefore, a low rate of spore formation. After this period of time, the turbidity was measured again in the MacFarland nephelometer, adjusting to a value of 3 with sterile saline.

The study of the activity of Samples 1 and 2 at 1% and 2% respectively was carried out in 96-well microtiter plates (ThermofisherR, Nunclon 96-Well MicroWell Plate w/Lid, Flat; 50/Cs, Denmark). For this, 20 μL of cell suspensions and 100 μL of sample 1 or sample 2 (1:6) were added to each of the wells. 100 μL of saline were added to positive growth controls. 1% bleach (control) was also used as a control for bactericidal activity. The efficacy of the different tested samples was evaluated after 5, 15 and 30 min at room temperature and under anaerobic conditions. After the corresponding time, the plates they were centrifuged at 2500 rpm for 15 minutes. The tested samples product was tapped off and the cells deposited at the bottom of the wells were resuspended with 150 μL of Brain Heart Infusion (BHI). The optical density (OD) of each of the wells at 600 nm (t=0 h) was immediately measured. After 48 h of incubation of the plates at 37° C. and in anaerobiosis, the OD was measured again at 600 nm (t=48 h).

The efficacy of the tested samples of the present invention on the vegetative forms of both ribotypes of C. difficile was evaluated by the difference in the increase in OD 600 nm at 48 h versus time 0 h (DO600/48−DO600/0).

The statistical analysis of the results was performed by the Kruskal Wallis test followed by Tukey's multiple comparison post-hoc test.

### Results

The more effective the product by reducing the number of vegetative cells, the smaller the increase in OD600 will be. In fact, negative values were detected when all the vegetable cells of C. difficile died.

The efficacy of the different tested compositions was evaluated by the increase in optical density at 600 nm (OD600) of C. difficile after its exposure for 5, 15 and 30 min with the tested samples 1 and 2 of the present invention and also with 1% bleach. On the other hand, the OD600 after 48 h of incubation of untreated C. difficile 027/078 (control) was also measured.

The efficient analysis of the tested compositions of the present invention expressed as DO600/48-DO600/0 for both ribotypes of C. difficile are disclosed in Table below:

| | DO600 nm_t48 h-DO600 nm_t0 h | | | | | |
|---|---|---|---|---|---|---|
| Tested | C. difficile 027 | | | C. difficile 078 | | |
| Samples | 5 min | 15 min | 30 min | 5 min | 15 min | 30 min |
| C1 (untreated) | 0.094 | 0.118 | 0.094 | 0.098 | 0.098 | 0.106 |
| Sample 1 (1%) | −0.011 | −0.061 | −0.074 | −0.028 | −0.047 | −0.068 |
| Sample 2 (2%) | −0.149 | −0.172 | −0.220 | −0.206 | −0.253 | −0.198 |
| C2 (bleach) | −0.019 | −0.004 | −0.006 | −0.011 | −0.005 | −0.015 |

For the untreated culture of C. difficile 027/078, an increase of OD600 until about 0.1 was observed. On the contrary, both compositions of the present invention (samples 1 and 2) significantly reduced (p<0.001) the proportion of vegetative cells of C. difficile with respect to an untreated control culture.

For the compositions of the present invention, both compositions (samples 1 and 2) significantly reduced the proportion of vegetative cells of C. difficile in comparison with the treatment with bleach. In particular, Sample 2 comprising 2% of the composition of Example 22 of the present invention is capable of statistically reducing the activity of C. difficile 027/078 after ONLY 5 min of exposure (p<0.05); and Sample 1 comprising 1% of the composition of Example 22 of the present invention is capable of statistically reducing the activity of C. difficile 027/078 after 15 min of exposure (p<0.05).

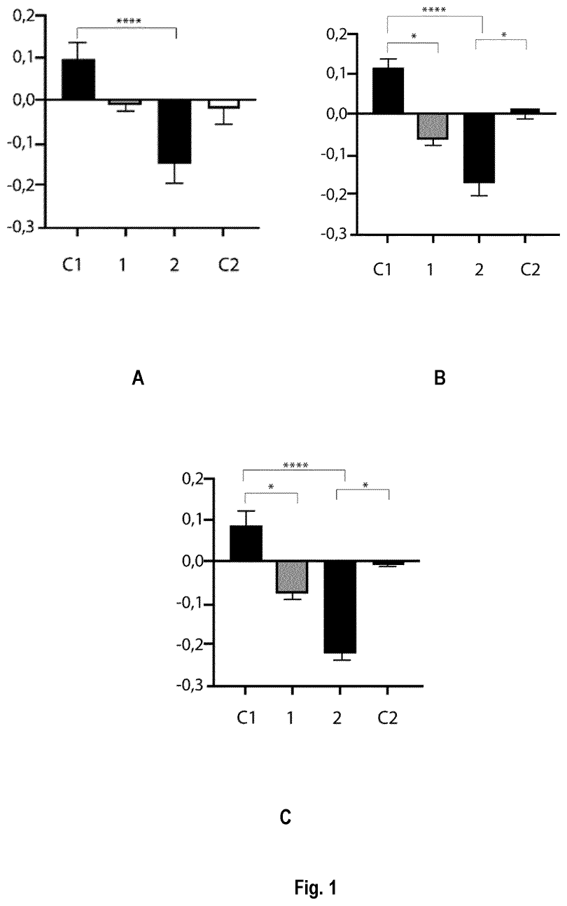

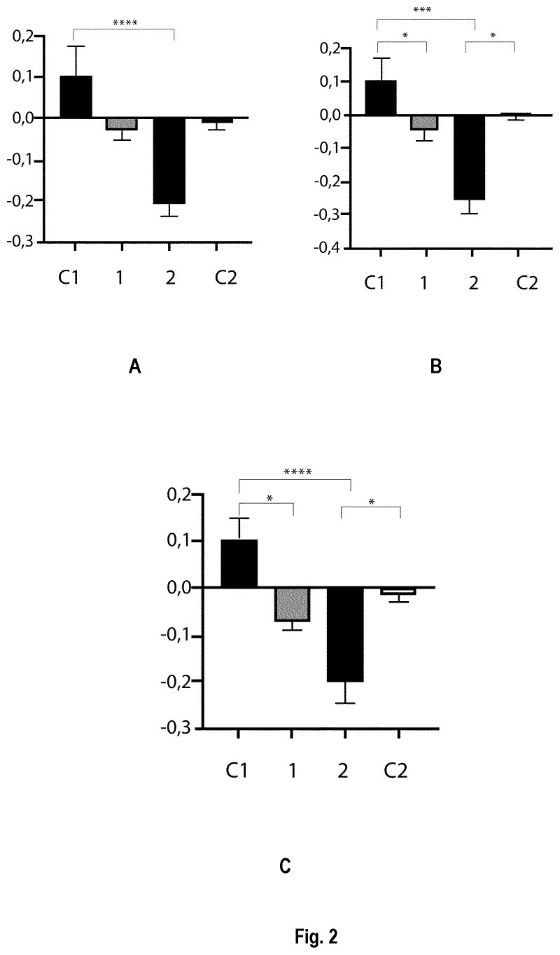

Furthermore, as it is shown in Table above, negative values of DO600 nm_t48 h-DO600 nm_t0 h were observed for the compositions of the invention. In fact, those values were more negative than those observed when bleach was used as biocide. It means that the compositions of the present invention have a biocidal efficiency against vegetative cells of C. difficile comparable to bleach at short exposure times and they have a higher efficiency against vegetative cells of C. difficile than bleach at longer exposure times to 15 min (cf. FIGS. 1 and 2).

Therefore, the compositions of the present invention are useful for the prevention and/or treatment of a disease or conditions cause by C. difficile, and as disinfection agent without causing surface (metal) corruption, inactivation by inorganic matter, skin, or mucous membranes irritation. Furthermore, the efficacy of the reduction of vegetative cells of C. difficile is also advantageous because inhibit the stimulation of sporulation. Finally, the stability of the compositions of the present invention allows having an enlarged useful life, reducing their application, and reducing the total healthcare cost of the treatment.

### 11. Tests of Fungicidal Activity Under the UNE-EN 13697 Standard

The results for the compositions of Examples 1, 8, 18, 21 (as disclosed in tables 1-3) were as follows. Assessment of fungicidal activity according to internal method based on UNE-EN 13697:2015 standard. Conclusion: The product complies with UNE-EN-13697 (fungicide) in dirty conditions, at concentrations of 0.2% in sterile water for the following fungi:
Puccinia hordei ATCC 22604
Puccinia striiformis ATCC PR-53
Septoria tritici ATCC 26518
Puccinia recondita ATCC PR-67
Puccinia graminis ATCC PR-99
Podosphaera pannosa
Botrytis ATCC 11542
Phytophtora cinnamoni Rands ATCC 46672
Pythium ATCC 24619
Pythiaceae ATCC 36433
Botryosphaeria corticola ATCC 60259
Dothideomycetes ATCC 6506
Ascomycota ATCC 18431
Xylariaceae ATCC 38988
Hypoxylon mediterraneum ATCC 38992
Agrobacterium tumefaciens ATCC 53385
Rhizopus ATCC 20577
Fusarium oxysporum ATCC 62506
Peronospora tabacina ATCC 3904
Phytophthora nicotinadiae ATCC 38606
Erysiphe necator ATCC 55 608 (powdery mildew)
Podosphaera ATCC 46 674 (powdery mildew)
Plasmopara viticula ATCC 27 294
Erysiphe necator ATCC 55 608 (powdery mildew)
Podosphaera ATCC 46 674 (powdery mildew)
Plasmopara viticula ATCC 27 294
Verticillium ATCC 16535

### 12—Bactericidal Activity Tests Under Standard UNE-EN 13697

The results for the compositions of Examples 1, 8, 18, 21 (as disclosed in tables 1-3) were as follows.

Assessment of bactericidal activity according to internal method based on UNE-EN 13697:2015 standard. Conclusion: The product complies with the UNE-EN-13697 standard (bactericidal) in dirty conditions, at concentrations of 0.2% in sterile water for the following bacteria:
Pseudomonas savastanoi ATCC 13522
Agrobacterium vitis ATCC 49767
Helicobacter pylori ATCC 700392

### 13—Antioxidant Activity Assay

The results for the compositions of Examples 1, 8, 18, 21 (as disclosed in tables 1-3) were as follows at a concentration of 100% (w/w). Antioxidant activity test carried out under the *ORAC (oxygen radical absorbance capacity) method with a result of 1322 umol Trolox equivalent/100 g.

In the ORAC method, a sample containing the substance to be tested is prepared. A source of free radicals is added to this sample, such as hydrogen peroxide (H₂O₂) or the free radical 2,2′-azobis(2-amidinopropane) dihydrochloride (AAPH). Next, the ability of the sample to inhibit oxidation induced by free radicals is measured. A fluorescent indicator is used that emits light when oxidized and is placed in the presence of the sample and free radicals. As the sample scavenges free radicals, oxidation of the fluorescent indicator is reduced and therefore light emission is reduced. Antioxidant activity is determined by measuring the decrease in fluorescence over time. The higher the ability of the sample to scavenge free radicals, the lower the fluorescence decay and the higher the antioxidant activity.

### 14—Bactericidal Activity Test at High pH Conditions

Test for bactericidal efficacy at high pH (pH9) using a sample having this pH.

The results for the compositions of Examples 1, 8, 18, 18, 21 (as disclosed in tables 1-3) and 1% (w/w) of Sodium Lauryl Sulfate to reach pH 9 were as follows:

Assessment of bactericidal activity according to internal method based on standard UNE-EN 13697:2015 Conclusions: The product complies with the UNE-EN-13697 standard (bactericide) in dirty conditions, at concentrations of 0.2% and 1% (w/w) of Sodium Lauryl Sulfate in sterile water at a contact time of 30 seconds.

### Common Mouth Bacterial Strains Used:

Escherichia coli ATCC 10 536
Staphylococcus aureus ATCC 6 538

### 15—Preservative Activity Test for Optimal pH Ranges for Increased Bacterial Growth

The results for the compositions of Examples 1, 8, 18, 21 (as disclosed in tables 1-3) were as follows at a concentration of 0.2% (w/w) in sterile water.

A study was carried out on bases for cosmetic products preserved with the product at pH 3.5 and pH 11.5.

The pH of both bases was brought to the optimum pH for bacterial growth to check the efficacy of the product as a preservative.

The study was be carried out on two different bases, one at pH 3.5 and the other at pH 11.5. To test the efficacy of the product as a preservative, both pHs were modified to an optimal range for bacterial growth. The optimum pH range for microbial growth was between 4.0-9.0 UpH.

The pH of the first sample (Sample 1) is increased to a value of 4.0 UpH using sodium bicarbonate. The pH of the second sample (Sample 2) is decreased to a value of 9.0 UpH by using citric acid.

Once the desired pH has been obtained, the samples are inoculated with strains of aerobic microorganisms and moulds and yeasts. In parallel, a positive control is carried out to check the effectiveness of the inoculation. After five minutes, the micro-organisms are checked both in the base samples and in the positive control.

All analyses will be carried out according to the Laboratory's internal procedures, each one based on its UNE-EN ISO reference. Likewise, all analyses are carried out under the Laboratory's internal quality control system.

Conclusion: After evaluating the results obtained in the control analyses carried out, it can be determined that the product is effective as a preservative against microbial growth.

### 16—Antimicrobial Activity Test Under American ASTM (American Society for Testing and Materials) Standards

Results for compositions of Examples 1, 8, 18, 21 (as disclosed in tables 1-3) were as follows at 0.2% (w/w) concentration in sterile water.

Evaluation of antimicrobial activity for water-miscible substances according to internal method based on ASTM E2783-22 (American Society for Testing and Materials)

Conclusions: The studied product presents a logarithmic reduction after 15 seconds of contact of 3.52 against Staphylococcus aureus ATCC 6538 and 3.49 against Escherichia coli ATCC 10 536.

The product presents a logarithmic reduction after 30 seconds of contact greater than 6 against to both microorganisms.

### 7—Nebulisation Efficacy Test

The results for the compositions of Examples 1, 8, 18, 21 (as disclosed in tables 1-3), were as follows: The objective of the test described in the following sections is to determine the environmental and surface antimicrobial action of the 0.2% in sterile water product applied by fogging.

After the initial sampling, the nebulisation of the 0.2% product is started. The nebulisation of the product is carried out in a room measuring 4.20×4.20×2.95 metres. The nebuliser equipment used, model KRDAP129-2B, has a nebulisation flow rate of 70 ml/min, a horizontal flow of 3 metres and a nebulisation particle size of 0-50 μM.

Successive ambient and surface controls are carried out after 5 minutes, 15 minutes and 30 minutes.

Sampling at the different times is carried out following the sampling method described in the initial sampling (T=0).

All samples taken at the end of the sampling process are kept refrigerated for preservation until arrival at the laboratory.

Once the sampling process has been completed, the micro-organisms of interest are tested in the laboratory. The analyses for the determination of the different micro-organisms are carried out by plate sowing on selective media, following UNE-EN 13697:2015.

Conclusion: In view of the results obtained, it can be concluded that the 0.2% product, applied by nebulisation under the conditions described in this report, has antimicrobial efficacy against the micro-organisms detected in this test, namely:
Bacillus cereus count
Coliform count
Enterobacteriaceae count
Escherichia coli count
Coagulase Staphylococci count
Mould and yeast counts
Total aerobic count at 30° C.
Total aerobic counts at 37° C.
Mesophilic aerobic counts
Faecal Streptococcus count
Detection of Listeria monocytogenes
Detection of Salmonella spp.
Enterohaemorrhagic Escherichia coli (EHD)
Brucella abortus
Brucella mellitensis
Brucella suis
Bacillus anthracis
Coixella burnetti
Echinoccus granulosus
Lestospira canicola
Leptospira icterohemorgrhagiae
Leptospira pomona
Chlamidya psitacci
Streptococcus pyogenes
Etisipelothrix rhusiopathiae
Campylobacter spp
Proteus mirabilis
Yersinia enterocolitica

### 18—Fog Efficiency Test

The results for the compositions of Examples 1, 8, 18, 21 (as disclosed in tables 1-3) were as follows at a concentration of 0.2% (w/w) plus 10.5% (w/w) Glycerine, 27% (w/w) Propylene Glycol and 61% (w/w) water. The objective of the test described in the following points is to determine the ambient and surface action of the product FOGSANIX applied by fog against Aerobes, Anaerobes, Escherichia coli, Listeria monocytogenes, Moulds and Yeasts.

An initial inoculation of the organism of interest is carried out in the environment and on the surface. After inoculation, an initial sampling is carried out before starting the fogging process with the product FOGSANIX, for which surface samples are taken with a sterile swab and environmental samples using a SAS MicroBio Air Sampler MBI Plus Serial number: 01111302.

A moistened swab is used for surface sampling, the swab is removed from its sterile wrapping and the tip is moistened by dipping it into a tube containing the diluent/neutraliser. Press the swab tip against the walls of the tube to remove excess diluent/neutraliser. Place the tips of the swabs on the surfaces to be examined at different heights (ground level, 1.50 m level, 2.5 m level and 4.5 m level) and swab an estimated area of about 100 cm² in each of the areas, while rotating the handle of each swab between thumb and forefinger, sampling in vertical and horizontal direction, e.g. 10 times in each direction.

The swabs are returned to each tube with diluent/neutraliser. The tubes are checked to ensure that the tubes are closed so that the swabs remain moist until analysis. For environmental sampling, the equipment is programmed for a sampling time of 2 min in which a sample is taken from 200 litres. At the end of the exposure time, the plate is removed from the equipment and stored in the oven for incubation.

After the initial sampling, the fogging of the FOGSANIX product is started with the fogging equipment supplied by 4Mediks with external dimensions 728 mm long, 428 mm wide and 719 mm high with voltage AC220-240, 50/60 hz 7 A. The fogging of the product is carried out in an industrial building of 34 m×27 m×7.5 m in total 6.885 m3, for 3 minutes 45 seconds and a maximum flow rate of 130 ml/minute. With a total test consumption of 487 ml. It is not necessary to rinse with water once the product has been applied.

Successive controls of both the environment and surfaces are carried out after 5 minutes, 10 minutes and 20 minutes. Sampling at the different times is carried out following the sampling method described in the initial sampling (T=0).

All samples taken at the end of the sampling process are kept refrigerated for preservation until analysis. Once the sampling process has been completed, the micro-organisms of interest are tested in the laboratory. The analyses are carried out by plate sowing on selective medium, following UNE-EN 13697:2015. Conclusions: In view of the results obtained, it can be concluded that the product FOGSANIX applied as a mist under the conditions described in this report has biocidal efficacy against the micro-organisms studied.

### 19—Hospital Clinic De Barcelona Trial to Establish MIC for Strains of Pseudomonas aeruginosa (ATCC 27853 and MB188) and Acinetobacter baumanii (ATCC 17978 and ATCC 19606)

The results for the compositions of Examples 1, 8, 18, 21 (as disclosed in tables 1-3) were as follows: Determination of the MIC of the product against control strains of Pseudomonas aeruginosa and Acinetobacter baumannii under identical experimental conditions: Exposure time (20 h-24 h) and temperature) (37°).

Based on the microdilution method in a 96-well microtiter plate as the reference method for MIC determination, cultures of 2 control strains of Pseudomonas aeruginosas (ATCC 27853 and MB188) and 2 control strains of Acinetobacter baumannii (ATCC 17978) were used. and ATCC 19606)

The culture medium chosen in the assay was Mueller Hinton II Broth (CAMH) which presents cation adjustment for calcium and magnesium ions, and is commonly used in sensitivity tests in aerobic Gram-negative and Gram-positive bacteria.

Taking into account that the approved concentration format for biocides is 0.2% (2000 μg/ml), 65,536 μg/ml and 128 μg/ml were estimated as the range of tested concentrations of the compound diluted in CAMH.

As an additional test, we wanted to verify the effectiveness of Tween20 in the biocide solution, so tests were carried out with the biocide compound and with the detergent compound (Tween 20), with a concentration of Tween20 twice the concentration of the biocide compound under study.

First, concentrations of 0.5 on the McFarland scale (1.5×108 CFU/ml) of each strain were obtained and a 1/1000 dilution was made in CAMH culture medium until reaching an initial plate concentration of the inoculum of approximately 1.5×105 CFU/ml

Regarding the microtiter plate method, 50 μg of CAMH medium were added to each well, followed by the addition of 50 μg of each compound to each of the control strains at the maximum concentration determined (65,536 μg/ml for the biocidal compound and 131,072 μg/ml for tween20) and serial dilutions were carried out in decreasing order until the chosen minimum concentration was reached (128 μg/ml for the biocidal compound and 256 μg/ml for Tween20).

Next, 50 μg of each strain were inoculated at an approximate concentration of 1.5×105 CFU/ml for each concentration of the compounds and the plates were incubated overnight without shaking at 37° C.

After the incubation time, each plate was read, considering the visualization of a button at the bottom of the well as a positive result, which would confirm the presence of bacterial growth.

| | biocide composition (μg/ml) |
|---|---|
| P. Aeruginosa ATCC 27853 | 512 |
| P. Aeruginosa MB188 | 1024 |
| A Baumannii ATCC 17978 | 256 |
| A Baumannii ATCC 19606 | 512 |

Conclusion: With the strains used and under the test conditions described, the product shows antimicrobial activity at concentrations lower than 2.000 μg/ml

### 20—Bactericidal Activity Test

Results for compositions of Example 22 (as disclosed in table 3) were as follows at 0.2% (w/w) concentration in sterile water.

Evaluation of bactericidal activity according to internal method based on UNE-EN 13697:2015.

Conclusions: The product complies with the UNE-EN-13697 standard (bactericide) in dirty conditions, at concentrations of 100%, 95% at a contact time of 5 minutes against:
Pseudomonas aeruginosa ATCC 15 442
Escherichia coli ATCC 10 536
Staphylococcus aureus ATCC 6 538
Enterococcus hirae ATCC 10 541

### 21—Fungicide Activity Test

Results for compositions of Example 22 (as disclosed in table 3) were as follows at 0.2% and 0.19% (w/w) concentration in sterile water.

Evaluation of fungal activity according to internal method based on UNE-EN 13697: 2015.

The product complies with the UNE-EN-13697 standard (fungal) in dirty conditions, at concentrations of 0.2% and 0.19% at a contact time of 5 minutes against:
Candida albicans ATCC 10231
Aspergillus brasiliensisb ATCC 16404

### 22—Antioxidant Activity Test

Results for compositions of Example 22 (as disclosed in table 3) were as follows at 100% (w/w) concentration Antioxidant activity test carried out under the ORAC (oxygen radical absorbance capacity) method with a result of 1251 umol Trolox equivalent/100 g.

The above results show that the compositions of the invention achieve a very high biocide activity despite containing low active ingredient concentrations. Moreover, it is important that these products entail a low toxicity, such that they can be registered via a simplified authorisation procedure such as EU Regulation No 528/2012.

The simplified authorisation procedure (EU Regulation No 528/2012) aims to promote the use of biocidal products that are less harmful to the environment and to human and animal health. For a biocidal product to be subject to a simplified authorisation procedure it must meet all of the following conditions:
the biocidal product does not contain any substance of concern.
the biocidal product does not contain nanomaterials
the biocidal product is sufficiently effective
the biocidal product does not require the use of personal protective equipment for its handling and its intended use

Having achieved a synergy between the different substances with a MIC of maximum 0.2% against all tested pathogens allows us to register a non-hazardous and non-toxic biocide product according to ECHA regulations (https://echa.europa.eu/es/regulations/biocidal-products-regulation/authorisation-of-biocidal-products/simplified-authorisation). This could not be achieved with any of the individual ingredients or combinations falling outside the scope of the present invention.

### Clauses

For reasons of completeness, various aspects of the invention are set out in the following numbered clauses:

Clause 1. A composition comprising:
(a) from 0.1% to 45% by weight of 5-Isopropil-2-metilfenol, or an extract comprising it;
(b) from 0.1% to 45% by weight of 1,3,3-Trimethyl-2-oxabicyclo[2.2.2]octane, or an extract comprising it;
(c) from 0.1% to 60% by weight of 4-Methyl-1-(propan-2-yl)cyclohex-3-en-1-ol, or an extract comprising it;
(d) from 0.1% to 45% by weight of 5-Methyl-2-(propan-2-yl) phenol; or an extract comprising it; and
(e) from 0.1% to 45% by weight of 5-Methyl-2-(propan-2-yl)cyclohexan-1-ol, or an extract comprising it,
(f) optionally, one or more additional active ingredients; and
(g) optionally, one or more appropriate acceptable excipients or carriers;
being the sum of ingredients up to 100% by weight.

Clause 2. The composition according to claim 1, comprising:
(a) from 0.5% to 45% by weight of 5-Isopropil-2-metilfenol, or an extract comprising it;
(b) from 0.5% to 42% by weight of 1,3,3-Trimethyl-2-oxabicyclo[2.2.2]octane, or an extract comprising it;
(c) from 10% to 57% by weight of 4-Methyl-1-(propan-2-yl)cyclohex-3-en-1-ol, or an extract comprising it;
(d) from 1% to 42% by weight of 5-Methyl-2-(propan-2-yl) phenol; or an extract comprising it;
(e) from 0.5% to 40% by weight of 5-Methyl-2-(propan-2-yl)cyclohexan-1-ol, or an extract comprising it;
(f) optionally, one or more additional active ingredients; and
(g) optionally, one or more appropriate acceptable excipients or carriers;
being the sum of ingredients up to 100% by weight;
particularly,
(a) from 1% to 43% by weight of 5-Isopropil-2-metilfenol, or an extract comprising it;
(b) from 1% to 40% by weight of 1,3,3-Trimethyl-2-oxabicyclo[2.2.2]octane, or an extract comprising it;
(c) from 15% to 55% by weight of 4-Methyl-1-(propan-2-yl)cyclohex-3-en-1-ol, or an extract comprising it;
(d) from 1% to 40% by weight of 5-Methyl-2-(propan-2-yl) phenol; or an extract comprising it; and
(e) from 0.5% to 35% by weight of 5-Methyl-2-(propan-2-yl)cyclohexan-1-ol, or an extract comprising it;
(f) optionally, one or more additional active ingredients; and
(g) optionally, one or more appropriate acceptable excipients or carriers;
being the sum of ingredients up to 100% by weight.

Clause 3. The composition according to any of the claims 1-2, comprising:
(a) from 2% to 43% by weight of 5-Isopropil-2-metilfenol, or an extract comprising it;
(b) from 2% to 35% by weight of 1,3,3-Trimethyl-2-oxabicyclo[2.2.2]octane, or an extract comprising it;
(c) from 17% to 54% by weight of 4-Methyl-1-(propan-2-yl)cyclohex-3-en-1-ol, or an extract comprising it;
(d) from 1.5% to 38% by weight of 5-Methyl-2-(propan-2-yl) phenol; or an extract comprising it; and
(e) from 1% to 30% by weight of 5-Methyl-2-(propan-2-yl)cyclohexan-1-ol, or an extract comprising it;
(f) optionally, one or more additional active ingredients; and
(g) optionally, one or more appropriate acceptable excipients or carriers;
being the sum of ingredients up to 100% by weight;
Particularly,
(a) from 3% to 43% by weight of 5-Isopropil-2-metilfenol, or an extract comprising it;
(b) from 3% to 32% by weight of 1,3,3-Trimethyl-2-oxabicyclo[2.2.2]octane, or an extract comprising it;
(c) from 20% to 53% by weight of 4-Methyl-1-(propan-2-yl)cyclohex-3-en-1-ol, or an extract comprising it;
(d) from 2% to 33% by weight of 5-Methyl-2-(propan-2-yl) phenol; or an extract comprising it; and
(e) from 1.5% to 20% by weight of 5-Methyl-2-(propan-2-yl)cyclohexan-1-ol, or an extract comprising it;
(f) optionally, one or more additional active ingredients; and
(g) optionally, one or more appropriate acceptable excipients or carriers;
being the sum of ingredients up to 100% by weight.

Clause 4. The composition according to any of the claims 1-3, wherein the one or more additional active ingredients is selected from the group consisting of antibiotic, viricide, fungicide, yeasticide, bactericide, skin conditioner, and prebiotic.

Clause 5. The composition according to claim 4, wherein the one or more additional active ingredients selected from the group consisting of organic acid containing compound; quaternary ammonium containing compound; methyl n-methyl anthranilate or an extract comprising it; guaiacol or an extract comprising it; p-cresol or an extract comprising it, cinnamaldehyde, geraniol, citral, methyl dihydrojasmonate (Hedione), 3-(3-propan-2-ylphenyl) butanal (florhydral), cis-para-mentanol (Cyclohexane methanol, 4-(1-methylethyl)-, cis-), and delta-damascone;
Particularly, selected from the group consisting of lactic acid, citric acid, hyaluronic acid, pyruvic acid, ferulic acid, benzalkonium chloride, dimethyl octadecyl[3-(trimethoxy silyl)propyl]ammonium chloride, methyl n-methyl anthranilate or an extract comprising it; guaiacol or an extract comprising it; p-cresol or an extract comprising it, cinnamaldehyde, geraniol, and citral.

Clause 6. The composition according to any of the claims 1-5, wherein the additional active ingredients are: A mixture of benzalkonium chloride and dimethyl octadecyl[3-(trimethoxy silyl)propyl]ammonium chloride; particularly from 5 to 20% by weight;
or alternatively,
a mixture of methyl n-methyl anthranilate, guaiacol, and p-cresol; particularly, from 0.1% to 25% by weight of methyl n-methyl anthranilate; from 0.1% to 25% by weight of guaiacol; and from 0.1% to 25% by weight of p-cresol;
or alternatively,
one or more selected from the group consisting of cinnamaldehyde, geraniol, citral; particularly from 5% to 20% by weight.

Clause 7. The composition according to any of the claims 1-6, wherein the one or more appropriate acceptable excipients or carriers is selected from the group consisting of surfactants, pH-regulating, gelling agent, solvents, co-solvents, rheological modifier agent, preservative, antioxidants, pH regulating agent, emulsifying agent, stabilizer, chelating agent, flavouring agent, fragrance, and perfume.

Clause 8. The composition according to any of the claims 1-7, which has a pH equal to or lower than 8; or alternatively, which has a pH higher than 8

Clause 9. Product comprising:
(i) an effective amount of a composition as defined in any of the claims 1-8; and
(ii) one or more appropriate acceptable excipients of carriers.

Clause 10. The product according to claim 9, comprising from 0.001% to 10% w/w of the composition as defined in any of the claims 1-8 in relation to the total weight of the product; particularly from 0.001% to 7% w/w; particularly from 0.001% to 5% w/w;
particularly from 0.001% to 4% w/w; particularly from 0.001% to 3% w/w; particularly from 0.01% to 3% w/w; particularly from 0.1% to 3% w/w; particularly from 0.2% to 3% w/w; and particularly from 0.01% to 2% w/w.

Clause 11. The composition as defined in any of the claims 1-8; or alternatively, the product as defined in any of the claims 9-10, which is selected from the group consisting of:
a pharmaceutical composition or product comprising (i) a therapeutically effective amount of a composition as defined in any of the claims 1-8; and (ii) one or more pharmaceutically acceptable excipients or carriers;
a veterinary composition or product comprising (i) a veterinary effective amount of a composition as defined in any of the claims 1-8; and (ii) one or more veterinary acceptable excipients or carriers;
a cosmetic composition or product comprising (i) a cosmetically effective amount of a composition as defined in any of the claims 1-8; and (ii) one or more cosmetically acceptable excipients or carriers;
a phytosanitary composition or product comprising (i) a phytosanitary effective amount of a composition as defined in any of the claims 1-8; and (ii) one or more agriculturally acceptable excipients or carriers;
a disinfectant composition or product comprising (i) a disinfectant effective amount of a composition as defined in any of the claims 1-8; and (ii) one or more appropriate acceptable excipients or carriers;
a dietary supplement composition or product comprising (i) an effective amount of a composition as defined in any of the claims 1-8; and (ii) one or more edible acceptable excipients or carriers;
a food/feed additive composition or product comprising (i) an effective amount of a composition as defined in any of the claims 1-8; and (ii) one or more edible acceptable excipients or carriers; and
a scented/perfumery composition or product comprising (i) appropriate amount of the composition as defined in any of the claims 1-8; and (ii) one or more appropriate excipients or carriers.

Clause 12. The product according to any of the claims 9-11, which is a pharmaceutical product for use in therapy or a veterinary product for use in veterinary,
Particularly, for use as biocide;
Particularly, for use in the treatment or prevention of bacterial, viral, fungal or yeast disease or condition; particularly, for use in the treatment or prevention of a bacterial, viral, fungal or yeast disease or condition caused by a pathogen selected from the group consisting of VPH (Human Papilloma Virus), HBV (Hepatitis B), Coronavirus, Herpes Virus, H7N9 (Influenza A), ECBO (Enterovirus Bovine), Rotavirus (viral Colitis), Vaccina Virus (smallpox), Polyoma Virus SV40, Bacteriophage for Lactobacillus, Poliovirus, Adenovirus, Norovirus (viral Colitis), Epstein-Barr, Polio virus type 1, LSc-2ab (Picornavirus), Adenovirus type 5, Strain Adenoid 75, ATCC VR-5, Murine norovirus, Strain S99 Berlin, Pseudomonas Aeruginosa, Escherichia Coli, Staphylococcus Aureus, Staphylococcus Epidermidis, Staphylococcus Hominis, Enterococcus Hirae, Burkholderia cepacia, Streptococcus mutans, Porphyromonas gingivalis, Fusobacterium nucleatum, Corynebacterium xerosis, Micrococcus spp, Lactobacillus acidophilus, Gardnerella vaginalis, Propionibacterium acnes, Legionella pneumophila, Proteus vulgaris, Salmonella (enteritis), Listeria monocytogenes, Fungal Spores (Teliospores, Zoospores, Ascospores, Zygospores), Bacillus, Clostridia, Xylella fastidiosa, Clostridium difficile, Erwinia amylovora, Xanthomonas arboricola pv. Pruni; Candida Albicans, Aspergillus Brasiliensis, Aspergillus Niger, Pityrosporum ovale, Campylobacter, Botrytis cinerea, Fusarium, Mildiu, Oidio, Phytophthora, Pythium, Fusarium oxysporum, Peronospora tabacina (tabaco), Phytophthora nicotinadiae (tabaco), Puccinia Hordei, Puccinia striiformis, Septoria tritici, Puccinia recondita, Puccinia graminis, Puccinia striiformis, Phytophthora cinnamoni Rands., Pythium spiculum, Pythium sterilum, Pythiaceae, Botryosphaeria corticola, dotidiomycete fungus, ascomycete fungus, Order Xylariales, Biscogniauxia mediterranea, Agrobacterium tumefaciens, Pseudomonas savastanoi, and Agrobacterium vitis.

Clause 13. Use of the product according to any of the claims 9-11, which is a cosmetic product in cosmetics; particularly, as skin care agent; more particularly, wherein the skin care comprises ameliorating at least one of the following symptoms: roughness, flakiness, dehydration, tightness, chapping, and lack of elasticity; particularly, as deodorant; and
particularly, as antioxidant and/or preservative agent.

Clause 14. Use of the product according to any of the claims 9-11, which is a phytosanitary product as phytosanitary biocide or booster agent;
particularly, for the treatment or prevention of a bacterial, viral, fungal, or yeast plant disease or condition; more particularly, for a plant disease or condition;
more particularly, for a plant and fruit trees caused by a pathogen selected from the group consisting of fire blight, bacterial spot of stone fruit trees and almond trees.

Clause 15. Use of the product according to any of the claims 9-11, which is:
a disinfectant product as disinfectant agent; particularly, as disinfectant of solid surfaces, spaces, and water;
or alternatively,
a food/feed additive product as food/feed preservative, food/feed antioxidant, food/feed antiseptic and food/feed biocide, and technological adjuvant.

### CITATION LIST

cf. Halldor Thormar, Lipids and Essential Oils as Antimicrobial Agents, Wiley Ed, edition 2011, chapter 9-11, pp. 203-262).
EP0244363
European Standard protocol UNE-EN 1276:2010
European Standard protocol UNE-EN-13697:2015
European Standard protocol UNE-EN 14476:2014+A1
European Standard protocol UNE-EN 1650:2008+A1
European Standard protocol UNE-EN 1500:2013
European Standard protocol UNE-EN 1650:2008
European Standard protocol UNE-EN 14476
ISO standard ISO 16212:2017
ISO standard ISO 22717:2016
ISO standard ISO 22718:2016
ISO standard ISO 21150:2016
ISO standard ISO 18416:2017
ISO standard ISO 21149:2017
American Society for Testing and Materials ASTM E2783-22
1European Standard protocol UNE-EN 1276:2020
EU Regulation No 528/2012
https://echa.europa.eu/es/regulations/biocidal-products-regulation/authorisation-of-biocidal-products/simplified-authorisation

1. A composition comprising:
(a) from 25% to 43% by weight of 5-Isopropil-2-metilfenol (carvacrol);
(b) from 20% to 38% by weight of 1,3,3-Trimethyl-2-oxabicyclo[2.2.2]octane (eucalyptol); and
(c) from 21% to 38% by weight of 4-Methyl-1-(propan-2-yl)cyclohex-3-en-1-ol (terpinen-4-ol);
being the sum of ingredients up to 100% by weight.

2. The composition according to claim 1, further comprising (d) from 1% to 9% by weight of 5-Methyl-2-(propan-2-yl) phenol (thymol).

3. The composition according to claim 1, further comprising (e) from 1% to 9% by weight of 5-Methyl-2-(propan-2-yl)cyclohexan-1-ol (menthol).

4. The composition according to claim 1, further comprising one or more additional active ingredients.

5. The composition according to claim 1, wherein the one or more additional active ingredient is selected from the group consisting of antibiotic, viricide, fungicide, yeasticide, bactericide, skin conditioner, and prebiotic.

6. The composition according to claim 5, wherein the one or more additional active ingredients is/are selected from the group consisting of organic acid containing compound; quaternary ammonium containing compound, such as quaternary ammonium silane salt; methyl n-methyl anthranilate, guaiacol, p-cresol, cinnamaldehyde, geraniol, citral, methyl dihydrojasmonate (Hedione), 3-(3-propan-2-ylphenyl)butanal (florhydral), cis-para-mentanol (Cyclohexane methanol, 4-(1-methylethyl)-, cis-), delta-damascone, eugenol, p-cymene, alfa-pinene, beta-pinene, beta-caryophyllene, caryophylene oxyde, terpinene, and terpinolene, particularly, selected from the group consisting of lactic acid, citric acid, hyaluronic acid, pyruvic acid, ferulic acid, benzalkonium chloride, dimethyl octadecyl[3-(trimethoxy silyl)propyl] ammonium chloride, methyl n-methyl anthranilate; guaiacol; p-cresol, cinnamaldehyde, geraniol, and citral.

7. The composition according to claim 4, wherein the additional active ingredients are:
A mixture of benzalkonium chloride and dimethyl octadecyl[3-(trimethoxy silyl)propyl] ammonium chloride; particularly from 5 to 20% by weight;
or alternatively,
a mixture of methyl n-methyl anthranilate, guaiacol, and p-cresol; particularly, from 0.1% to 25% by weight of methyl n-methyl anthranilate; from 0.1% to 25% by weight of guaiacol; and
from 0.1% to 25% by weight of p-cresol;
or alternatively,
one or more selected from the group consisting of cinnamaldehyde, geraniol, citral; particularly from 5% to 20% by weight.

8. The composition according to claim 1, further comprising one or more appropriate acceptable excipients or carriers.

9. The composition according to claim 8, wherein the one or more appropriate acceptable excipients or carriers is/are selected from the group consisting of surfactants, pH-regulating, gelling agent, solvents, co-solvents, rheological modifier agent, preservative, antioxidants, pH regulating agent, emulsifying agent, stabilizer, chelating agent, flavouring agent, fragrance, and perfume.

10. The composition according to claim 1 consisting of the ingredients as defined in claim 1.

11. The composition according to claim 1, wherein:
(a) the concentration of carvacrol is from 30 to 40% by weight;
(b) the concentration of eucalyptol is from 25 to 36% by weight;
(c) the concentration of terpinen-4-ol is from 25 to 36% by weight;
(d) the concentration of thymol, if present, is from 1 to 6% by weight; and
(e) the concentration of menthol, if present, is from 2 to 5% by weight.

12. The composition according to claim 11, wherein:
(a) the concentration of carvacrol is from 30 to 35% by weight;
(b) the concentration of eucalyptol is from 31 to 36% by weight; and
(c) the concentration of terpinen-4-ol is from 31 to 36% by weight.

13. The composition according to claim 11, wherein:
(a) the concentration of carvacrol is from 35 to 40% by weight;
(b) the concentration of eucalyptol is from 25 to 30% by weight;
(c) the concentration of terpinen-4-ol is from 25 to 30% by weight;
(d) the concentration of thymol, if present, is from 3 to 6% by weight; and
(e) the concentration of menthol, if present, is from 2 to 5% by weight.

14. Product comprising:
(i) 0.001% to 50% w/w of a composition as defined in claim 1 in relation to the total weight of the product, particularly from 0.01% to 2% of a composition as defined in claim 1 in relation to the total weight of the product, more particularly from 0.01% to 0.5% of a composition as defined in claim 1 in relation to the total weight of the product; and
(ii) one or more appropriate acceptable excipients of carriers.

15. The composition as defined in claim 1, which is selected from the group consisting of:
a pharmaceutical composition or product comprising (i) a therapeutically effective amount of a composition as defined in claim 1; and (ii) one or more pharmaceutically acceptable excipients or carriers;
a veterinary composition or product comprising (i) a veterinary effective amount of a composition as defined in claim 1; and (ii) one or more veterinary acceptable excipients or carriers;
a cosmetic composition or product comprising (i) a cosmetically effective amount of a composition as defined in claim 1; and (ii) one or more cosmetically acceptable excipients or carriers;
a phytosanitary composition or product comprising (i) a phytosanitary effective amount of a composition as defined in claim 1; and (ii) one or more agriculturally acceptable excipients or carriers;
a disinfectant composition or product comprising (i) a disinfectant effective amount of a composition as defined in claim 1; and (ii) one or more appropriate acceptable excipients or carriers;
a dietary supplement composition or product comprising (i) an effective amount of a composition as defined in claim 1; and (ii) one or more edible acceptable excipients or carriers;
a food/feed additive composition or product comprising (i) an effective amount of a composition as defined in claim 1; and (ii) one or more edible acceptable excipients or carriers; and
a scented/perfumery composition or product comprising (i) appropriate amount of the composition as defined in claim 1; and (ii) one or more appropriate excipients or carriers.

16. A method for preparing a product according to claim 14, said method comprising mixing a composition as defined in claim 1 with one or more appropriate acceptable excipients of carriers.

17. The product according to claim 14, which is a pharmaceutical product for use in therapy or a veterinary product for use in veterinary, particularly, for use as biocide;
particularly, for use in the treatment or prevention of bacterial, viral, fungal or yeast disease or condition;
particularly, for use in the treatment or prevention of a bacterial, viral, fungal or yeast disease or condition caused by a pathogen selected from the group consisting of VPH (Human Papilloma Virus), HBV (Hepatitis B), Coronavirus, Herpes Virus, H7N9 (Influenza A), ECBO (Enterovirus Bovine), Rotavirus (viral Colitis), Vaccina Virus (smallpox), Polyoma Virus SV40, Bacteriophage for Lactobacillus, Poliovirus, Adenovirus, Norovirus (viral Colitis), Epstein-Barr, Polio virus type 1, LSc-2ab (Picornavirus), Adenovirus type 5, Strain Adenoid 75, ATCC VR-5, Murine norovirus, Strain S99 Berlin, Pseudomonas Aeruginosa, Escherichia Coli, Staphylococcus Aureus, Staphylococcus Epidermidis, Staphylococcus Hominis, Enterococcus Hirae, Burkholderia cepacia, Streptococcus mutans, Porphyromonas gingivalis, Fusobacterium nucleatum, Corynebacterium xerosis, Micrococcus spp, Lactobacillus acidophilus, Gardnerella vaginalis, Propionibacterium acnes, Legionella pneumophila, Proteus vulgaris, Salmonella (enteritis), Listeria monocytogenes, Fungal Spores (Teliospores, Zoospores, Ascospores, Zygospores), Bacillus, Clostridia, Xylella fastidiosa, Clostridium difficile, Erwinia amylovora, Xanthomonas arboricola pv. Pruni; Candida Albicans, Aspergillus Brasiliensis, Aspergillus niger, Pityrosporum ovale, Campylobacter, Botrytis cinerea, Fusarium, Mildiu, Oidio, Phytophthora, Pythium, Fusarium oxysporum, Peronospora tabacina (tabaco), Phytophthora nicotinadiae (tabaco), Puccinia Hordei, Puccinia striiformis, Septoria tritici, Puccinia recondita, Puccinia graminis, Puccinia striiformis, Phytophthora cinnamoni Rands., Pythium spiculum, Pythium sterilum, Pythiaceae, Botryosphaeria corticola, dotidiomycete fungus, ascomycete fungus, Order Xylariales, Biscogniauxia mediterranea, Agrobacterium tumefaciens, Pseudomonas savastanoi, and Agrobacterium vitis.

18. Use of the product according to claim 14, which is a cosmetic product in cosmetics;
particularly, as skin care agent; more particularly, wherein the skin care comprises ameliorating at least one of the following symptoms: roughness, flakiness, dehydration, tightness, chapping, and lack of elasticity;
particularly, as deodorant; and
particularly, as antioxidant and/or preservative agent.

19. Use of the product according to claim 14, which is a phytosanitary product as phytosanitary biocide or booster agent;
particularly, for the treatment or prevention of a bacterial, viral, fungal, or yeast plant disease or condition; more particularly, for a plant disease or condition;
more particularly, for a plant and fruit trees caused by a pathogen selected from the group consisting of fire blight, bacterial spot of stone fruit trees and almond trees.

20. Use of the product according to claim 14, which is:
a disinfectant product as disinfectant agent; particularly, as disinfectant of solid surfaces, spaces, and water,
or alternatively,
a food/feed additive product as food/feed preservative, food/feed antioxidant, food/feed antiseptic and food/feed biocide, and technological adjuvant.
